(19) United States
(12) Patent Application Publication
(10) Pub. No.: US 2004/0116922 A1
(43) Pub. Date: Jun. 17, 2004

(54) Methods and apparatus for treating intervertebral discs

(75) Inventors: David C. Hovda, Mountain View, CA (US); Brian E. Martini, Menlo Park, CA (US); Allison C. Johnson, San Mateo, CA (US); Norman R. Sanders, Hillsborough, CA (US)

Correspondence Address: ARTHROCARE CORPORATION, 680 VAQUEROS AVENUE, SUNNYVALE, CA, 94085-3523, US

(73) Assignee: ArthroCare Corporation, Sunnyvale, CA

(21) Appl. No.: 10/656,597

(22) Filed: Sep. 5, 2003

Related U.S. Application Data
(60) Provisional application No. 60/408,967, filed on Sep. 5, 2002.

(51) Int. Cl. A61B 18/18

(52) U.S. Cl. 606/41

(57) ABSTRACT

The systems, devices and methods described herein selectively apply electrical energy and thermal energy to structures within a patient's body, such as the intervertebral disc. The systems, devices and methods thereof are useful for shrinkage, ablation, and/or hemostasis of tissue and other body structures in open and endoscopic spine surgery. In particular the systems, devices and methods described herein provide advantages in the treatment of disc tissue. The system, devices and methods described herein also treat a disc via a microdiscectomy which require little or no annulotomy.

### BACKGROUND OF THE INVENTION

The present invention relates to the field of electrosurgery, and more particularly to surgical devices and methods which employ high-frequency electrical energy to treat soft tissue in regions of the spine. The present invention also relates to improved devices and methods for the treatment of intervertebral discs

Intervertebral discs mainly function to articulate and cushion the vertebrae, while the interspinous tissue (i.e., tendons and cartilage, and the like) function to support the vertebrae so as to provide flexibility and stability to the patient's spine.

The discs comprise a nucleus pulposus which is a central hydrophilic cushion. The nucleus is surrounded by an annulus fibrosus or annulus which is a multi-layered fibrous ligament. The disc also includes vertebral endplates which are located between the disc and adjacent vertebrae.

The nucleus pulposus occupies 25-40% of the total disc cross-sectional area. It is composed mainly of mucoid material containing mainly proteoglycans with a small amount of collagen. The proteoglycans consist of a protein core having attached chains of negatively charged keratin sulphate and chondroitin sulphate. Such a structure is the reason the nucleus pulposus is a “loose or amorphous hydrogel” which has the capacity to bind water and usually contains 70-90% water by weight.

The annulus fibrosus forms the outer boundary of the disc and is composed of highly structured collagen fibers embedded in amorphous base substance also composed of water and proteoglycans. However, the amorphous base of the annulus is lower in content than in the nucleus. The collagen fibers of the annulus are arranged in concentric laminated bands. In each laminated band the fibers are parallel and attached to the adjacent vertebral bodies at roughly a 30° angle from the horizontal plane of the disc in both directions. There is a steady increase in the proportion of collagen from the inner to the outer annulus.

Each disc has two vertebral end-plates composed of hyaline cartilage. As mentioned above, the end-plates separate the disc from adjacent vertebral bodies. The end-plates acts as a transitional zone between the harder bony vertebral bodies and the soft disc. Because the nucleus pulposus does not contain blood vessels (i.e., it is avascular), the disc receives most nutrients through the end-plate areas.

Many patients suffer from discogenic pain resulting from degenerative disc disease and/or vertebral disc herniation. Degeneration of discs occurs when they lose their water content and height, causing adjoining vertebrae to move closer together. The deterioration of the disc results in a decrease of the shock-absorbing ability of the spine. This condition also causes a narrowing of the neural openings in the sides of the spine which may pinch these nerves. Thus disc degeneration may eventually cause severe chronic and disabeling back and leg pain.

Disc herniations generally fall into three types of categories: 1) contained disc herniation (also known as contained disc protrusion); 2) extruded disc herniation; and 3) sequestered disc herniation (also known as a free fragment.)

In a contained herniation, a portion of the disc protrudes or bulges from a normal boundary of the disc. However, in a contained herniation, the nucleus pulposus and the disc does not breach the annulus fibrosus, rather a protrusion of the disc might mechanically compress and/or chemically irritate an adjacent nerve root. This condition leads to radicular pain, commonly referred to as sciatica (leg pain.) In an extruded herniation, the annulus is disrupted and a segment of the nucleus protrudes/extrudes from the disc. However in this condition, the nucleus within the disc remains contiguous with the extruded fragment. With a sequestered disc herniation, a nucleus fragment separates from the nucleus and disc.

Degenerating or injured discs may have weaknesses in the annulus contributing to herniation of the disc. The weakened annulus may allow fragments of nucleus pulposus to migrate through the annulus fibrosus and into the spinal canal. Once in the canal, the displaced nucleus pulposus tissue, or the protruding annulus may impinge on spinal nerves or nerve roots. A weakened annulus may also result in bulging (e.g., a contained herniation) of the disc. Mechanical compression and/or chemical irritation of the nerve may occur depending on the proximity of the bulge to a nerve. A patient with these conditions may experience pain, sensory, and motor deficit.

A significant percentage of such patients undergo surgical procedures to treat the disorders described above. These procedures include both percutaneous and open discectomy, and spinal fusion.

Often, symptoms from disc herniation can be treated successfully by non-surgical means, such as rest, therapeutic exercise, oral anti-inflammatory medications or epidural injection of corticosteroids. Such treatments result in a gradual but progressive improvement in symptoms and allow the patient to avoid surgical intervention.

In some cases, the disc tissue is irreparably damaged, thereby necessitating removal of a portion of the disc or the entire disc to eliminate the source of inflammation and pressure. In more severe cases, the adjacent vertebral bodies must be stabilized following excision of the disc material to avoid recurrence of the disabling back pain. One approach to stabilizing the vertebrae, termed spinal fusion, is to insert an interbody graft or implant into the space vacated by the degenerative disc. In this procedure, a small amount of bone may be grafted and packed into the implants. This allows the bone to grow through and around the implant, fusing the vertebral bodies and preventing reoccurrence of the symptoms.

Until recently, surgical spinal procedures resulted in major operations and traumatic dissection of muscle and bone removal or bone fusion. However, the development of minimally invasive spine surgery overcomes many of the disadvantages of traditional traumatic spine surgery. In endoscopic spinal procedures, the spinal canal is not violated and therefore epidural bleeding with ensuing scarring is minimized or completely avoided. In addition, the risk of instability from ligament and bone removal is generally lower in endoscopic procedures than with open procedures. Further, more rapid rehabilitation facilitates faster recovery and return to work.

Percutaneous techniques for the treatment of herniated discs include: chemonucleolysis; laser techniques; and mechanical techniques, such as automated percutaneous lumbar discectomy. These procedures generally require the surgeon to place an introducer needle or cannula from the external surface of the patient to the spinal disc(s) for passage of surgical instruments or device. Open techniques for the treatment of herniated discs involve surgical dissection through soft tissue and removal of a portion of vertebral bone. Conventionally, upon encountering the annulus a complex surgical incision, called an annulotomy, must be made to allow access of instruments into the disc so that decompress the disc may take place. Mechanical instruments, such as pituitary rongeurs, curettes, graspers, cutters, drills, microdebriders and the like are often used to remove the nucleus material. Unfortunately, these mechanical instruments greatly lengthen and increase the complexity of the procedure. In addition, and most significantly, the annulotomy itself may lead to future re-herniation of the disc or even accelerate disc degeneration. Discussion of the problems associated with the annulotomy are found in journals and other medical publications. (see e.g., Ahlgren, et al. Annular incision technique on the strength and multidirectional flexibility of the healing intervertebral disc., Spine Apr. 15, 1994; 9(8) pp 948-954; Ahlgren, et al. Effect of annular repair on the healing strength of the intervertebral disc: a sheep model., Spine Sep. 1, 2000; 25(17): pp 2167-2170.)

Previously, in order to prevent re-herniation of the annulus after performance of an annulotomy, the surgeon removes an excess amount of nucleus material from the disc to effect a pressure release for the site of the annulotomy. However, it was found that removing an excess amount of the nucleus pulposus destabilizes the disc leading to accelerated disc degeneration. See e.g., Meakin et al., The Effect of Partial Removal of the Nucleus Pulposus from the Intervertebral Disc on the Response of the Human Annulus Fibrosus to Compression., Clin Biomech (Bristol, Avon) February 2001; 16(2) pp. 121-128.

Monopolar and bipolar radiofrequency devices have been used in limited roles in spine surgery, primarily for hemostasis. Monopolar devices, however, suffer from the disadvantage that the electric current will flow through undefined paths in the patient's body, thereby increasing the risk of undesirable electrical stimulation to portions of the patient's body. In addition, since the defined path through the patient's body has a relatively high impedance (because of the large distance or resistivity of the patient's body), large voltage differences must typically be applied between the return and active electrodes in order to generate a current suitable for ablation or cutting of the target tissue. This current, however, may inadvertently flow along body paths having less impedance than the defined electrical path, which will substantially increase the current flowing through these paths, possibly causing damage to or destroying surrounding tissue or neighboring peripheral nerves.

Another significant disadvantage of conventional RF devices, particularly monopolar devices, is that the device causes nerve stimulation and interference with nerve monitoring equipment in the operating room. In addition, these devices typically operate by creating a voltage difference between the active electrode and the target tissue, causing an electrical arc to form across the physical gap between the electrode and tissue. At the point of contact of the electric arcs with tissue, rapid tissue heating occurs due to high current density between the electrode and tissue. This high current density increases the temperature of the cells causing cellular fluids to rapidly vaporize into steam, thereby producing a “cutting effect” by exploding the cells along the pathway of localized tissue heating. Thus, while the tissue parts along the pathway of evaporated cellular fluid, the heating process induces undesirable thermal collateral tissue damage in regions surrounding the target tissue site. This collateral tissue damage often includes indiscriminate destruction of tissue, resulting in thermal necrosis and the loss of the proper function of the tissue. In addition, the conventional device does not remove any tissue directly, but rather depends on destroying a zone of tissue and allowing the body to either encapsulate the zone with scar tissue or eventually remove the destroyed tissue via phagocytosis absorption.

A further problem with lasers and conventional RF devices is that the conduction of heat may cause unintentional damage to the vertebral end-plates. The vertebral end-plates contain chondrocytes which extract plasma and other nutrients from adjacent micro-capillaries to maintain the essential moisture and biochemistry within the disc. However, these chondrocytes are heat sensitive. Therefore, thermally damaging these chondrocytes may also destroy or impair the function of the vertebral end-plates thereby causing premature disc deterioration. In addition, damage of the end-plates may cause the adjacent formation of necrotic tissue, and/or thermal bone necrosis (i.e., a layer of dead bone), thereby creating a barrier to the passage of water and nutrients from the endplate into the disc. Such a condition may further accelerate the degeneration of the disc. The existence of necrotic tissue may also present problems if a fusion procedure is subsequently required. Any necrotic tissue at the site of the area to be fused must be removed or destroyed prior to fusion. Accordingly, the presence of necrotic tissue increases the duration of the fusion procedure and may adversely affect the outcome of the procedure.

Presently, there is a need for an improved treatment for individuals having disorders or abnormalities of an intervertebral disc. There is also a need for a minimally invasive treatment of intervertebral discs in order to alleviate the pain associated with disc disease. Such pain often being chronic, and debilitating. Furthermore, there is a need for a solution that overcomes the problems associated with an annulotomy performed on a disc during conventional surgery.

The methods and devices aimed at meeting the above needs should be applicable to all types of degenerative discs, and all levels of the vertebral column, including cervical, thoracic, and lumbar spine. Such methods and devices should also be applicable to all types of herniations.

### SUMMARY OF THE INVENTION

The present invention provides systems, apparatus, and methods for selectively applying electrical energy and thermal energy to structures within a patient's body, such as the intervertebral disc. The systems and methods of the present invention are useful for shrinkage, ablation, resection, aspiration, and/or hemostasis of tissue and other body structures in open and endoscopic spine surgery. In particular, the present invention includes a method and system for debulking, ablating, coagulating, and shrinking of disc tissue. The present invention also includes method and devices to treat a disc via a microdiscectomy which require little or no annulotomy. It is believed that use of the inventive methods as described herein provides for treatment of a herniated disc while retaining the integrity of the annulus of the disc to prevent or minimize the recurrence of subsequent herniation.

In one aspect, the present invention provides a method of treating a herniated intervertebral disc. The method comprising advancing an access device into the disc by expanding and/or separating layers of an annulus of the disc, advancing a treatment device into the disc using the access device, and activating the treatment device to treat the disc, wherein upon subsequent removal of the access device the separated layers of the annulus substantially close to seal the disc.

The activating of the treatment device includes positioning at least one active electrode within the intervertebral disc. High frequency voltage is applied between the active electrode(s) and one or more return electrode(s) to debulk, ablate, coagulate and/or shrink at least a portion of the nucleus pulposus and/or annulus. The high frequency voltage effects a controlled depth of thermal heating to reduce the water content of the nucleus pulposus, thereby debulking the nucleus pulposus and reducing the internal pressure on the annulus fibrosus. The treatment device may be expanded from a reduced profile to an expanded profile so that it enters the disc through a minimal opening or passageway and later expands to apply treatment within the disc.

In accordance with the procedure, energy may be applied to the outer annulus adjacent to the opening created by the access device to assist in closure of the annulus. The energy may be applied prior to, during, or subsequent to entry of the access device into the annulus. Energy may also be applied to an interior surface of the opening created by the access device.

In an exemplary embodiment, an electrically conductive media, such as isotonic saline or an electrically conductive gel, is delivered to the target site within the intervertebral disc prior to delivery of the high frequency energy. The conductive media will typically fill the entire target region such that the active electrode(s) are submerged throughout the procedure. In other embodiments, the extracellular conductive medium (e.g., the nucleus pulposus) in the patient's disc may be used as a substitute for, or as a supplement to, the electrically conductive media that is applied or delivered to the target site. For example, in some embodiments, an initial amount of conductive media is provided to initiate the requisite conditions for ablation. After initiation, the conductive medium already present in the patient's tissue is used to sustain these conditions.

In another aspect, the present invention provides a method of treating a disc having a contained herniation or fissure. The method comprises introducing an electrosurgical instrument into the patient's intervertebral disc either percutaneously or through an open procedure. The instrument is steered or otherwise guided into close proximity to the contained herniation or fissure and a high frequency voltage is applied between an active electrode and a return electrode so as to debulk the nucleus pulposus adjacent the contained herniation or fissure. In some embodiments a conductive medium is delivered into the intervertebral disc prior to applying the high frequency voltage to ensure that sufficient conductive medium exists for plasma formation and to conduct electric current between the active and return electrodes. Alternatively, the conductive medium can be delivered to the target site during the procedure. The heating delivered through the electrically conductive medium debulks the nucleus pulposus, and reduces the pressure on the annulus fibrosus so as to reduce the pressure on the affected nerve root and alleviate neck and back pain.

In another aspect, the present invention provides a method for treating degenerative intervertebral discs. The active electrode(s) are advanced into the target disc tissue in an ablation mode, where the high frequency voltage is sufficient to ablate or remove the nucleus pulposus through molecular dissociation or disintegration processes. In these embodiments, the high frequency voltage applied to the active electrode(s) is sufficient to vaporize an electrically conductive medium (e.g., gel, saline and/or intracellular fluid) between the active electrode(s) and the tissue. Within the vaporized fluid, an ionized plasma is formed and charged particles (e.g., electrons) cause the molecular breakdown or disintegration of several cell layers of the nucleus pulposus. This molecular dissociation is accompanied by the volumetric removal of the tissue. This process can be precisely controlled to effect the volumetric removal of tissue as thin as 10 microns to 150 microns with minimal heating of, or damage to, surrounding or underlying tissue structures. A more complete description of this phenomenon is described in commonly assigned U.S. Pat. No. 5,697,882 the complete disclosure of which is incorporated herein by reference.

The invention also includes an electrosurgical device for use with a high-frequency power supply, the device comprising, a shaft having a proximal portion and a distal portion, a return electrode at the distal portion of the shaft and having a return electrode surface area, the return electrode distally terminating in a tip portion, at least one active electrode at the distal portion of the shaft, and having an active electrode surface area, the active electrode further comprising an arm portion being radially spaced from the return electrode, wherein the tip portion of the return electrode is distally spaced from the arm portion of the active electrode, and a connector located at the proximal portion of the shaft and adapted to couple the return electrode and each active electrode to respective poles of the high-frequency power supply.

Variations of the device include devices where the return electrode surface area is greater than the active electrode surface area.

Variations also include devices where a portion of the return electrode comprises at least one segment having a raised surface, whereby the raised surfaces increase the return electrode surface area. The raised surface may be a conductive member (e.g., a wire) that is coiled around a body portion of the return electrode.

Variations of the device include active electrodes having arm portions, wherein at least the arm portion of the active electrode is adapted to deform such that the device may assume a reduced profile. The arm portion may be elastically deformable, or the arm portion may comprise a shape memory alloy such that the arm portion of the active electrode may return from the reduced profile upon application of heat.

The active electrodes of the present invention may have arm portions which comprise at least one section of reduced surface area adapted to produce a high current density.

The invention also includes a system for treating tissue with a high-frequency power supply, the system comprising: a source of electrically conductive medium, an electro surgical device for use with the high-frequency power supply, the device comprising, a shaft having a proximal portion and a distal portion, a return electrode at the distal portion of the shaft and having a return electrode surface area, the return electrode having a tip portion, at least one active electrode at the distal portion of the shaft, and having an active electrode surface area, the active electrode further comprising an arm portion being radially spaced from the return electrode, wherein the tip portion of the return electrode is distally spaced from the arm portion of the active electrode, and a connector located at the proximal portion of the shaft and adapted to couple the return electrode and each active electrode to respective poles of the high-frequency power supply; and wherein the source of electrically conductive medium provides an electrically conductive path which completes a circuit between the return electrode and the active electrode.

### BRIEF DESCRIPTION OF THE DRAWINGS

FIG. 1 is a view of an example of an electrosurgical system incorporating a power supply and an electrosurgical probe of the present invention;

FIG. 2 schematically illustrates one embodiment of a power supply according to the present invention;

FIG. 3 illustrates an electrosurgical system incorporating a plurality of active electrodes and associated current limiting elements;

FIGS. 4A-4B illustrate side views of electrosurgical probes according to the present invention;

FIGS. 5A, 5C-5E illustrate variations of an electrosurgical device of the present invention, FIG. 5B illustrates an example of a multi-lumen shaft of the present invention;

FIGS. 6A-6D illustrate additional variations of return electrodes of the present invention;

FIG. 7 illustrates a cross sectional view of an outer sheath or covering located about the shaft and electrodes to restrain the electrodes in a reduced profile;

FIG. 8 is an illustration of an ablative zone created by a variation of a probe of the invention;

FIGS. 9A-9B illustrate additional variations of active electrodes for use with the present invention;

FIG. 9C illustrates a side view of an active electrode;

FIGS. 10A-10H illustrate examples of cross sections of active electrodes;

FIGS. 11A-11B illustrate an example of another probe for use with the inventive procedure;

FIG. 12 illustrates an example of electrical connections for coupling active electrode(s) and a return electrode in a variation of the present invention;

FIG. 13A is an illustration of a herniated disc prior to treatment with the invention described herein;

FIGS. 13B-13D illustrate an example of accessing a herniated disc to treat with the inventive procedure; and

FIGS. 14A-14F illustrate treatment of a herniated disc in accordance with the devices and methods of the present invention.

### DESCRIPTION OF SPECIFIC EMBODIMENTS

The present invention provides systems and methods for selectively applying electrical energy to a target location within or on a patient's body, particularly including support tissue or other body structures in the spine. These procedures include treating interspinous tissue, degenerative discs, laminectomy/discectomy procedures for treating herniated discs, decompressive laminectomy for stenosis in the lumbosacral and cervical spine, localized tears or fissures in the annulus, nucleotomy, disc fusion procedures, medial facetectomy, posterior lumbosacral and cervical spine fusions, treatment of scoliosis associated with vertebral disease, foraminotomies to remove the roof of the intervertebral foramina to relieve nerve root compression and anterior cervical and lumbar discectomies. These procedures may be performed through open procedures, or using minimally invasive techniques, such as thoracoscopy, arthroscopy, laparoscopy or the like.

The present invention involves techniques for treating disc abnormalities with RF energy. In some embodiments, RF energy is used to ablate, debulk and/or stiffen the tissue structure of the disc to reduce the volume of the disc, thereby relieving neck and back pain. In one aspect of the invention, spinal disc tissue is volumetrically removed or ablated to form one or more voids, holes, channels, divots, or other spaces within the disc. In this procedure, a high frequency voltage difference is applied between one or more active electrode(s) and one or more return electrode(s) to develop high electric field intensities in the vicinity of the target tissue. The high electric field intensities adjacent to the active electrode(s) lead to electric field induced molecular breakdown of target tissue through molecular dissociation (rather than thermal evaporation or carbonization.) Applicant believes that the tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to dehydrating the tissue material by the removal of liquid within the cells of the tissue and extracellular fluids, as is typically the case with electrosurgical desiccation and vaporization.

The present invention also involves a system and method for treating the interspinous tissue (e.g., tendons, cartilage, synovial tissue in between the vertebrae, and other support tissue within and surrounding the vertebral column.) In some embodiments, RF energy is used to heat and shrink the interspinous tissue to stabilize the vertebral column and reduce pain in the back and neck. In one aspect of the invention, an active electrode is positioned adjacent the interspinous tissue and the interspinous tissue is heated, preferably with RF energy, to a sufficient temperature to shrink the interspinous tissue. In a specific embodiment, a high frequency voltage difference is applied between one or more active electrode(s) and one or more return electrode(s) to develop high electric field intensities in the vicinity of the target tissue to controllably heat the target tissue.

The high electric field intensities may be generated by applying a high frequency voltage that is sufficient to vaporize an electrically conductive medium over at least a portion of the active electrode(s) in the region between the distal tip of the active electrode(s) and the target tissue. The electrically conductive medium may be a liquid or gas, such as isotonic saline, blood, extracelluar or intracellular fluid, delivered to, or already present at, the target site, or a viscous fluid, such as a gel, applied to the target site. Since the vapor layer or vaporized region has a relatively high electrical impedance, it minimizes the current flow into the electrically conductive medium. This ionization, under the conditions described herein, induces the discharge of energetic electrons and photons from the vapor layer and to the surface of the target tissue. A more detailed description of this phenomena, termed Coblation® can be found in commonly assigned U.S. Pat. No. 5,697,882 the complete disclosure of which is incorporated herein by reference.

Applicant believes that the principle mechanism of tissue removal in the Coblation® mechanism of the present invention is energetic electrons or ions that have been energized in a plasma adjacent to the active electrode(s.) When a liquid is heated enough that atoms vaporize off the surface faster than they recondense, a gas is formed. When the gas is heated enough that the atoms collide with each other and knock their electrons off in the process, an ionized gas or plasma is formed (the so-called “fourth state of matter”.) A more complete description of plasma can be found in Plasma Physics, by R. J. Goldston and P. H. Rutherford of the Plasma Physics Laboratory of Princeton University (1995), the complete disclosure of which is incorporated herein by reference. When the density of the vapor layer (or within a bubble formed in the electrically conducting liquid) becomes sufficiently low (i.e., less than approximately 10²⁰ atoms/cm³ for aqueous solutions), the electron mean free path increases to enable subsequently injected electrons to cause impact ionization within these regions of low density (i.e., vapor layers or bubbles.) Once the ionic particles in the plasma layer have sufficient energy, they accelerate towards the target tissue. Energy evolved by the energetic electrons (e.g., 3.5 eV to 5 eV) can subsequently bombard a molecule and break its bonds, dissociating a molecule into free radicals, which then combine into final gaseous or liquid species.

Plasmas may be formed by heating a gas and ionizing the gas by driving an electric current through it, or by shining radio waves into the gas. Generally, these methods of plasma formation give energy to free electrons in the plasma directly, and then electron-atom collisions liberate more electrons, and the process cascades until the desired degree of ionization is achieved. Often, the electrons carry the electrical current or absorb the radio waves and, therefore, are hotter than the ions. Thus, in applicant's invention, the electrons, which are carried away from the tissue towards the return electrode, carry most of the plasma's heat with them, allowing the ions to break apart the tissue molecules in a substantially non-thermal manner.

In some embodiments, the present invention applies high frequency (RF) electrical energy in an electrically conducting media environment to shrink or remove (i.e., resect, cut, or ablate) a tissue structure and to seal transected vessels within the region of the target tissue. The present invention may also be useful for sealing larger arterial vessels, e.g., on the order of about 1 mm in diameter. In some embodiments, a high frequency power supply is provided having an ablation mode, wherein a first voltage is applied to an active electrode sufficient to effect molecular dissociation or disintegration of the tissue, and a coagulation mode, wherein a second, lower voltage is applied to an active electrode (either the same or a different electrode) sufficient to heat, shrink, and/or achieve hemostasis of severed vessels within the tissue. In other embodiments, an electrosurgical instrument is provided having one or more coagulation electrode(s) configured for sealing a severed vessel, such as an arterial vessel, and one or more active electrodes configured for either contracting the collagen fibers within the tissue or removing (ablating) the tissue, e.g., by applying sufficient energy to the tissue to effect molecular dissociation. In the latter embodiments, the coagulation electrode(s) may be configured such that a single voltage can be applied to coagulate with the coagulation electrode(s), and to ablate or shrink with the active electrode(s.) In other embodiments, the power supply is combined with the coagulation instrument such that the coagulation electrode is used when the power supply is in the coagulation mode (low voltage), and the active electrode(s) are used when the power supply is in the ablation mode (higher voltage.)

In another aspect, the present invention may be used to shrink or contract collagen connective tissue which support the vertebral column or connective tissue within the disc. In these procedures, the RF energy heats the tissue directly by virtue of the electrical current flow therethrough, and/or indirectly through the exposure of the tissue to fluid heated by RF energy, to elevate the tissue temperature from normal body temperatures (e.g., 37° C.) to temperatures in the range of 45° C. to 90° C., preferably in the range from about 60° C. to 70° C. Thermal shrinkage of collagen fibers occurs within a small temperature range which, for mammalian collagen is in the range from 60° C. to 70° C. (Deak, G., et al., “The Thermal Shrinkage Process of Collagen Fibres as Revealed by Polarization Optical Analysis of Topooptical Staining Reactions,” Acta Morphological Acad. Sci. of Hungary, Vol. 15(2), pp. 195-208, 1967.) Previously reported research has attributed thermal shrinkage of collagen to the cleaving of the internal stabilizing cross-linkages within the collagen matrix (Deak, ibid.) It has also been reported that when the collagen temperature is increased above 70° C., the collagen matrix begins to relax again and the shrinkage effect is reversed resulting in no net shrinkage (Allain, J. C., et al., “Isometric Tensions Developed During the Hydrothermal Swelling of Rat Skin,” Connective Tissue Research, Vol. 7, pp. 127-133, 1980), the complete disclosure of which is incorporated by reference. Consequently, the controlled heating of tissue to a precise depth is critical to the achievement of therapeutic collagen shrinkage. A more detailed description of collagen shrinkage can be found in commonly assigned U.S. Pat. No. 6,159,194, the complete disclosure of which is incorporated by reference.

The preferred depth of heating to effect the shrinkage of collagen in the heated region (i.e., the depth to which the tissue is elevated to temperatures between 60° C. to 70° C.) generally depends on (1) the thickness of the target tissue, (2) the location of nearby structures (e.g., nerves) that should not be exposed to damaging temperatures, and/or (3) the location of the collagen tissue layer within which therapeutic shrinkage is to be effected. The depth of heating is usually in the range from 1.0 mm to 5.0 mm. In some embodiments of the present invention, the tissue is purposely damaged in a thermal heating mode to create necrosed or scarred tissue at the tissue surface. The high frequency voltage in the thermal heating mode is below the threshold of ablation as described above, but sufficient to cause some thermal damage to the tissue immediately surrounding the electrodes without vaporizing or otherwise debulking this tissue in situ. Typically, it is desired to achieve a tissue temperature in the range of about 60° C. to 100° C. to a depth of about 0.2 mm to 5 mm, usually about 1 mm to 2 mm. The voltage required for this thermal damage will partly depend on the electrode configurations, the conductivity of the area immediately surrounding the electrodes, the time period in which the voltage is applied and the depth of tissue damage desired. The higher the voltage, the less time required. If the voltage is too high, however, the surface tissue may be vaporized, debulked or ablated, which is generally undesirable.

The present invention is also useful for removing or ablating tissue around nerves, such as spinal, peripheral or cranial nerves. One of the significant drawbacks with the prior art shavers or microdebriders, conventional electrosurgical devices and lasers is that these devices do not differentiate between the target tissue and the surrounding nerves or bone. Therefore, the surgeon must be extremely careful during these procedures to avoid damage to the bone or nerves within and around the target site. In the present invention, the Coblation® process for removing tissue results in extremely small depths of collateral tissue damage as discussed above. This allows the surgeon to remove tissue close to a nerve without causing collateral damage to the nerve fibers.

In addition to the generally precise nature of the novel mechanisms of the present invention, applicant has discovered an additional method of ensuring that adjacent nerves are not damaged during tissue removal. According to the present invention, systems and methods are provided for distinguishing between the fatty tissue immediately surrounding nerve fibers and the normal tissue that is to be removed during the procedure. Peripheral nerves usually comprise a connective tissue sheath, or epineurium, enclosing the bundles of nerve fibers, each bundle being surrounded by its own sheath of connective tissue (the perineurium) to protect these nerve fibers. The outer protective tissue sheath or epineurium typically comprises a fatty tissue (e.g., adipose tissue) having substantially different electrical properties than the normal target tissue, such as the turbinates, polyps, mucus tissue or the like, that are, for example, removed from the nose during sinus procedures. The system of the present invention measures the electrical properties of the tissue at the tip of the probe with one or more active electrode(s.) These electrical properties may include electrical conductivity at one, several or a range of frequencies (e.g., in the range from 1 kHz to 100 MHz), dielectric constant, capacitance or combinations of these. In this embodiment, an audible signal may be produced when the sensing electrode(s) at the tip of the probe detects the fatty tissue surrounding a nerve, or direct feedback control can be provided to only supply power to the active electrode(s) either individually or to the complete array of electrodes, if and when the tissue encountered at the tip or working end of the probe is normal tissue based on the measured electrical properties.

In one embodiment, the current limiting elements (discussed in detail below) are configured such that the active electrodes will shut down or turn off when the electrical impedance reaches a threshold level. When this threshold level is set to the impedance of the fatty tissue surrounding nerves, the active electrodes will shut off whenever they come in contact with, or in close proximity to, nerves. Meanwhile, the other active electrodes, which are in contact with or in close proximity to tissue, will continue to conduct electric current to the return electrode. This selective ablation or removal of lower impedance tissue in combination with the Coblation® mechanism of the present invention allows the surgeon to precisely remove tissue around nerves or bone. Applicant has found that the present invention is capable of volumetrically removing tissue closely adjacent to nerves without impairment the function of the nerves, and without significantly damaging the tissue of the epineurium. One of the significant drawbacks with the prior art microdebriders, conventional electrosurgical devices and lasers is that these devices do not differentiate between the target tissue and the surrounding nerves or bone. Therefore, the surgeon must be extremely careful during these procedures to avoid damage to the bone or nerves within and around the nasal cavity. In the present invention, the Coblation® process for removing tissue results in extremely small depths of collateral tissue damage as discussed above. This allows the surgeon to remove tissue close to a nerve without causing collateral damage to the nerve fibers.

In addition to the above, applicant has discovered that the Coblation® mechanism of the present invention can be manipulated to ablate or remove certain tissue structures, while having little effect on other tissue structures. As discussed above, the present invention uses a technique of vaporizing electrically conductive medium to form a plasma layer or pocket around the active electrode(s), and then inducing the discharge of energy from this plasma or vapor layer to break the molecular bonds of the tissue structure. Based on initial experiments, applicants believe that the free electrons within the ionized vapor layer are accelerated in the high electric fields near the electrode tip(s.) When the density of the vapor layer (or within a bubble formed in the electrically conducting liquid) becomes sufficiently low (i.e., less than approximately 10²⁰ atoms/cm³ for aqueous solutions), the electron mean free path increases to enable subsequently injected electrons to cause impact ionization within these regions of low density (i.e., vapor layers or bubbles.) Energy evolved by the energetic electrons (e.g., 4 eV to 5 eV) can subsequently bombard a molecule and break its bonds, dissociating a molecule into free radicals, which then combine into final gaseous or liquid species.

The energy evolved by the energetic electrons may be varied by adjusting a variety of factors, such as: the number of active electrodes; electrode size and spacing; electrode surface area; asperities and sharp edges on the electrode surfaces; electrode materials; applied voltage and power; current limiting means, such as inductors; electrical conductivity of the fluid in contact with the electrodes; density of the fluid; and other factors. Accordingly, these factors can be manipulated to control the energy level of the excited electrons. Since different tissue structures have different molecular bonds, the present invention can be configured to break the molecular bonds of certain tissue, while having too low an energy to break the molecular bonds of other tissue. For example, fatty tissue, (e.g., adipose) tissue has double bonds that require a substantially higher energy level than 4 eV to 5 eV to break (typically on the order of about 8 eV.) Accordingly, the present invention in its current configuration generally does not ablate or remove such fatty tissue. However, the present invention may be used to effectively ablate cells to release the inner fat content in a liquid form. Of course, factors may be changed such that these double bonds can also be broken in a similar fashion as the single bonds (e.g., increasing voltage or changing the electrode configuration to increase the current density at the electrode tips.) A more complete description of this phenomena can be found in commonly assigned U.S. Pat. No. 6,355,032, the complete disclosure of which is incorporated herein by reference.

In yet other embodiments, the present invention provides systems, apparatus and methods for selectively removing tumors, e.g., facial tumors, or other undesirable body structures while minimizing the spread of viable cells from the tumor. Conventional techniques for removing such tumors generally result in the production of smoke in the surgical setting, termed an electrosurgical or laser plume, which can spread intact, viable bacterial or viral particles from the tumor or lesion to the surgical team or to other portions of the patient's body. This potential spread of viable cells or particles has resulted in increased concerns over the proliferation of certain debilitating and fatal diseases, such as hepatitis, herpes, HIV and papillomavirus. In the present invention, high frequency voltage is applied between the active electrode(s) and one or more return electrode(s) to volumetrically remove at least a portion of the tissue cells in the tumor through the dissociation or disintegration of organic molecules into non-viable atoms and molecules. Specifically, the present invention converts the solid tissue cells into non-condensable gases that are no longer intact or viable, and thus, not capable of spreading viable tumor particles to other portions of the patient's brain or to the surgical staff. The high frequency voltage is preferably selected to effect controlled removal of these tissue cells while minimizing substantial tissue necrosis to surrounding or underlying tissue. A more complete description of this phenomena can be found in commonly assigned U.S. Pat. Nos. 6,149,120 and 6,296,136, the complete disclosures of which are incorporated herein by reference.

The electrosurgical probe or catheter of the present invention can comprise a shaft or a handpiece having a proximal end and a distal end which supports one or more active electrode(s.) The shaft or handpiece may assume a wide variety of configurations, with the primary purpose being to mechanically support the active electrode and permit the treating physician to manipulate the electrode from a proximal end of the shaft. The shaft may be rigid or flexible, with flexible shafts optionally being combined with a generally rigid external tube for mechanical support. Flexible shafts may be combined with pull wires, shape memory actuators, and other known mechanisms for effecting selective deflection of the distal end of the shaft to facilitate positioning of the electrode array. The shaft will usually include a plurality of wires or other conductive elements running axially therethrough to permit connection of the electrode array to a connector at the proximal end of the shaft.

For endoscopic procedures within the spine, the shaft will have a suitable diameter and length to allow the surgeon to reach the target site (e.g., a disc or vertebra) by delivering the shaft through the thoracic cavity, the abdomen or the like. Thus, the shaft will usually have a length in the range of about 5.0 cm to 30.0 cm, and a diameter in the range of about 0.2 mm to about 20 mm. Alternatively, the shaft may be delivered directly through the patient's back in a posterior approach, which would considerably reduce the required length of the shaft. In any of these embodiments, the shaft may also be introduced through rigid or flexible endoscopes. Alternatively, the shaft may be a flexible catheter that is introduced through a percutaneous penetration in the patient. Specific shaft designs will be described in detail in connection with the figures hereinafter.

In an alternative embodiment, the probe may comprise a long, thin needle (e.g., on the order of about 1 mm in diameter or less) that can be percutaneously introduced through the patient's back directly into the spine. The needle will include one or more active electrode(s) for applying electrical energy to tissues within the spine. The needle may include one or more return electrode(s), or the return electrode may be positioned on the patient's back, as a dispersive pad. In either embodiment, sufficient electrical energy is applied through the needle to the active electrode(s) to either shrink the collagen fibers within the spinal disc, to ablate tissue within the disc, or to shrink support fibers surrounding the vertebrae.

The electrosurgical instrument may also be a catheter that is delivered percutaneously and/or endoluminally into the patient by insertion through a conventional or specialized guide catheter, or the invention may include a catheter having an active electrode or electrode array integral with its distal end. The catheter shaft may be rigid or flexible, with flexible shafts optionally being combined with a generally rigid external tube for mechanical support. Flexible shafts may be combined with pull wires, shape memory actuators, and other known mechanisms for effecting selective deflection of the distal end of the shaft to facilitate positioning of the electrode or electrode array. The catheter shaft will usually include a plurality of wires or other conductive elements running axially therethrough to permit connection of the electrode or electrode array and the return electrode to a connector at the proximal end of the catheter shaft. The catheter shaft may include a guide wire for guiding the catheter to the target site, or the catheter may comprise a steerable guide catheter. The catheter may also include a substantially rigid distal end portion to increase the torque control of the distal end portion as the catheter is advanced further into the patient's body. Specific shaft designs will be described in detail in connection with the figures hereinafter.

The active electrode(s) are preferably supported within or by an inorganic insulating support positioned near the distal end of the instrument shaft. The return electrode may be located on the instrument shaft, on another instrument or on the external surface of the patient (i.e., a dispersive pad.) The close proximity of nerves and other sensitive tissue in and around the spinal cord, however, makes a bipolar design more preferable because this minimizes the current flow through non-target tissue and surrounding nerves. Accordingly, the return electrode is preferably either integrated with the instrument body, or another instrument located in close proximity thereto. The proximal end of the instrument(s) will include the appropriate electrical connections for coupling the return electrode(s) and the active electrode(s) to a high frequency power supply, such as an electrosurgical generator.

In some embodiments, the active electrode(s) have an active portion or surface with surface geometries shaped to promote the electric field intensity and associated current density along the leading edges of the electrodes. Suitable surface geometries may be obtained by creating electrode shapes that include preferential sharp edges, or by creating asperities or other surface roughness on the active surface(s) of the electrodes. Electrode shapes according to the present invention can include the use of formed wire (e.g., by drawing round wire through a shaping die) to form electrodes with a variety of cross-sectional shapes, such as square, rectangular, L or V shaped, or the like. Electrode edges may also be created by removing a portion of the elongate metal electrode to reshape the cross-section. For example, material can be ground along the length of a round or hollow wire electrode to form D or C shaped wires, respectively, with edges facing in the cutting direction. Alternatively, material can be removed at closely spaced intervals along the electrode length to form transverse grooves, slots, threads or the like along the electrodes.

Additionally or alternatively, the active electrode surface(s) may be modified through chemical, electrochemical or abrasive methods to create a multiplicity of surface asperities on the electrode surface. These surface asperities will promote high electric field intensities between the active electrode surface(s) and the target tissue to facilitate ablation or cutting of the tissue. For example, surface asperities may be created by etching the active electrodes with etchants having a pH less than 7.0 or by using a high velocity stream of abrasive particles (e.g., grit blasting) to create asperities on the surface of an elongated electrode. A more detailed description of such electrode configurations can be found in U.S. Pat. No. 5,843,019, the complete disclosure of which is incorporated herein by reference.

The return electrode is typically spaced proximally from the active electrode(s) a suitable distance to avoid electrical shorting between the active and return electrodes in the presence of electrically conductive medium. In most of the embodiments described herein, the distal edge of the exposed surface of the return electrode is spaced about 0.5 mm to 25 mm from the proximal edge of the exposed surface of the active electrode(s), preferably about 1.0 mm to 5.0 mm. Of course, this distance may vary with different voltage ranges, conductive mediums, and depending on the proximity of tissue structures to active and return electrodes. The return electrode will typically have an exposed length in the range of about 1 mm to 20 mm.

The current flow path between the active electrodes and the return electrode(s) may be generated by submerging the tissue site in an electrical conducting fluid (e.g., within a viscous fluid, such as an electrically conductive gel) or by directing an electrically conductive medium along a path to the target site (i.e., a liquid, such as isotonic saline, hypotonic saline or a gas, such as argon.) The conductive gel may also be delivered to the target site to achieve a slower more controlled delivery rate of conductive medium. In addition, the viscous nature of the gel may allow the surgeon to more easily contain the gel around the target site (e.g., rather than attempting to contain isotonic saline.) A more complete description of an exemplary method of directing electrically conductive medium between the active and return electrodes is described in U.S. Pat. No. 5,697,281, previously incorporated herein by reference. Alternatively, the body's natural conductive tissues, such as blood or extracellular saline, may be sufficient to establish a conductive path between the return electrode(s) and the active electrode(s), and to provide the conditions for establishing a vapor layer, as described above. However, conductive medium that is introduced into the patient is usually preferred over blood because blood will tend to coagulate at certain temperatures. In addition, the patient's blood may not have sufficient electrical conductivity to adequately form a plasma in some applications. Advantageously, a liquid electrically conductive medium (e.g., isotonic saline) may be used to concurrently “bathe” the target tissue surface to provide an additional means for removing any tissue, and to cool the region of the target tissue ablated in the previous moment.

The power supply, or generator, may include a fluid interlock for interrupting power to the active electrode(s) when there is insufficient conductive medium around the active electrode(s.) This ensures that the instrument will not be activated when conductive medium is not present, minimizing the tissue damage that may otherwise occur. A more complete description of such a fluid interlock can be found in commonly assigned, U.S. Pat. No. 6,235,020, the complete disclosure of which is incorporated herein by reference.

The present invention may use a single active electrode or an array of active electrodes spaced around the distal surface of a catheter or probe. In the latter embodiment, the electrode array usually includes a plurality of independently current-limited and/or power-controlled active electrodes to apply electrical energy selectively to the target tissue while limiting the unwanted application of electrical energy to the surrounding tissue and environment resulting from power dissipation into surrounding electrically conductive mediums, such as blood, normal saline, and the like. The active electrodes may be independently current-limited by isolating the terminals from each other and connecting each terminal to a separate power source that is isolated from the other active electrodes. Alternatively, the active electrodes may be connected to each other at either the proximal or distal ends of the catheter to form a single wire that couples to a power source.

In one configuration, each individual active electrode in the electrode array is electrically insulated from all other active electrodes in the array within said instrument and is connected to a power source which is isolated from each of the other active electrodes in the array or to circuitry which limits or interrupts current flow to the active electrode when low resistivity material (e.g., blood, electrically conductive saline irrigant or electrically conductive gel) causes a lower impedance path between the return electrode and the individual active electrode. The isolated power sources for each individual active electrode may be separate power supply circuits having internal impedance characteristics which limit power to the associated active electrode when a low impedance return path is encountered. By way of example, the isolated power source may be a user selectable constant current source. In this embodiment, lower impedance paths will automatically result in lower resistive heating levels since the heating is proportional to the square of the operating current times the impedance. Alternatively, a single power source may be connected to each of the active electrodes through independently actuatable switches, or by independent current limiting elements, such as inductors, capacitors, resistors and/or combinations thereof. The current limiting elements may be provided in the instrument, connectors, cable, controller, or along the conductive path from the controller to the distal tip of the instrument. Alternatively, the resistance and/or capacitance may occur on the surface of the active electrode(s) due to oxide layers which form selected active electrodes (e.g., titanium or a resistive coating on the surface of metal, such as platinum.)

It should be clearly understood that the invention is not limited to electrically isolated active electrodes, or even to a plurality of active electrodes. For example, the array of active electrodes may be connected to a single lead that extends through the catheter shaft to a power source of high frequency current.

The electrically conductive medium should have a threshold conductivity to provide a suitable conductive path between the return electrode and the active electrode(s.) The electrical conductivity of the fluid (in units of millisiemens per centimeter or mS/cm) will usually be greater than 0.2 mS/cm, preferably will be greater than 2 mS/cm and more preferably greater than 10 mS/cm. In an exemplary embodiment, the electrically conductive medium is isotonic saline, which has a conductivity of about 17 mS/cm. Applicant has found that a more conductive medium, or one with a higher ionic concentration, will usually provide a more aggressive ablation rate. For example, a saline solution with higher levels of sodium chloride than conventional saline (which is on the order of about 0.9% sodium chloride) e.g., on the order of greater than 1% or between about 3% and 20%, may be desirable. Alternatively, the invention may be used with different types of conductive mediums that increase the power of the plasma layer by, for example, increasing the quantity of ions in the plasma, or by providing ions that have higher energy levels than sodium ions. For example, the present invention may be used with elements other than sodium, such as potassium, magnesium, calcium and other metals near the left end of the periodic chart. In addition, other electronegative elements may be used in place of chlorine, such as fluorine.

The voltage difference applied between the return electrode(s) and the active electrode(s) will be at high or radio frequency, typically between about 5 kHz and 20 MHz, usually being between about 30 kHz and 2.5 MHz, preferably being between about 50 kHz and 500 kHz, often less than 350 kHz, and often between about 100 kHz and 200 kHz. In some applications, applicant has found that a frequency of about 100 kHz is useful because the tissue impedance is much greater at this frequency. In other applications, such as procedures in or around the heart or head and neck, higher frequencies may be desirable (e.g., 400-600 kHz) to minimize low frequency current flow into the heart or the nerves of the head and neck. The RMS (root mean square) voltage applied will usually be in the range from about 5 volts to 1000 volts, preferably being in the range from about 10 volts to 500 volts, often between about 150 volts to 400 volts depending on the active electrode size, the operating frequency and the operation mode of the particular procedure or desired effect on the tissue (i.e., contraction, coagulation, cutting or ablation.) Typically, the peak-to-peak voltage for ablation or cutting with a square wave form will be in the range of 10 volts to 2000 volts and preferably in the range of 100 volts to 1800 volts and more preferably in the range of about 300 volts to 1500 volts, often in the range of about 300 volts to 800 volts peak to peak (again, depending on the electrode size, number of electrons, the operating frequency and the operation mode.) Lower peak-to-peak voltages will be used for tissue coagulation, thermal heating of tissue, or collagen contraction and will typically be in the range from 50 to 1500, preferably 100 to 1000 and more preferably 120 to 400 volts peak-to-peak (again, these values are computed using a square wave form.) Higher peak-to-peak voltages, e.g., greater than about 800 volts peak-to-peak, may be desirable for ablation of harder material, such as bone, depending on other factors, such as the electrode geometries and the composition of the conductive medium.

As discussed above, the voltage is usually delivered in a series of voltage pulses or alternating current of time varying voltage amplitude with a sufficiently high frequency (e.g., on the order of 5 kHz to 20 MHz) such that the voltage is effectively applied continuously (as compared with e.g., lasers claiming small depths of necrosis, which are generally pulsed about 10 Hz to 20 Hz.) In addition, the duty cycle (i.e., cumulative time in any one-second interval that energy is applied) is on the order of about 50% for the present invention, as compared with pulsed lasers which typically have a duty cycle of about 0.0001%.

The preferred power source of the present invention delivers a high frequency current selectable to generate average power levels ranging from several milliwatts to tens of watts per electrode, depending on the volume of target tissue being treated, and/or the maximum allowed temperature selected for the instrument tip. The power source allows the user to select the voltage level according to the specific requirements of a particular neurosurgery procedure, cardiac surgery, arthroscopic surgery, dermatological procedure, ophthalmic procedures, open surgery or other endoscopic surgery procedure. For cardiac procedures and potentially for neurosurgery, the power source may have an additional filter, for filtering leakage voltages at frequencies below 100 kHz, particularly voltages around 60 kHz. Alternatively, a power source having a higher operating frequency, e.g., 300 kHz to 600 kHz may be used in certain procedures in which stray low frequency currents may be problematic. A description of one suitable power source can be found in commonly assigned U.S. Pat. Nos. 6,142,992 and 6,235,020, the complete disclosure of both patents are incorporated herein by reference for all purposes.

The power source may be current limited or otherwise controlled so that undesired heating of the target tissue or surrounding (non-target) tissue does not occur. In a presently preferred embodiment of the present invention, current limiting inductors are placed in series with each independent active electrode, where the inductance of the inductor is in the range of 10 uH to 50,000 uH, depending on the electrical properties of the target tissue, the desired tissue heating rate and the operating frequency. Alternatively, capacitor-inductor (LC) circuit structures may be employed, as described previously in U.S. Pat. No. 5,697,909, the complete disclosure of which is incorporated herein by reference. Additionally, current limiting resistors may be selected. Preferably, these resistors will have a large positive temperature coefficient of resistance so that, as the current level begins to rise for any individual active electrode in contact with a low resistance medium (e.g., saline irrigant or blood), the resistance of the current limiting resistor increases significantly, thereby minimizing the power delivery from said active electrode into the low resistance medium (e.g., saline irrigant or blood.)

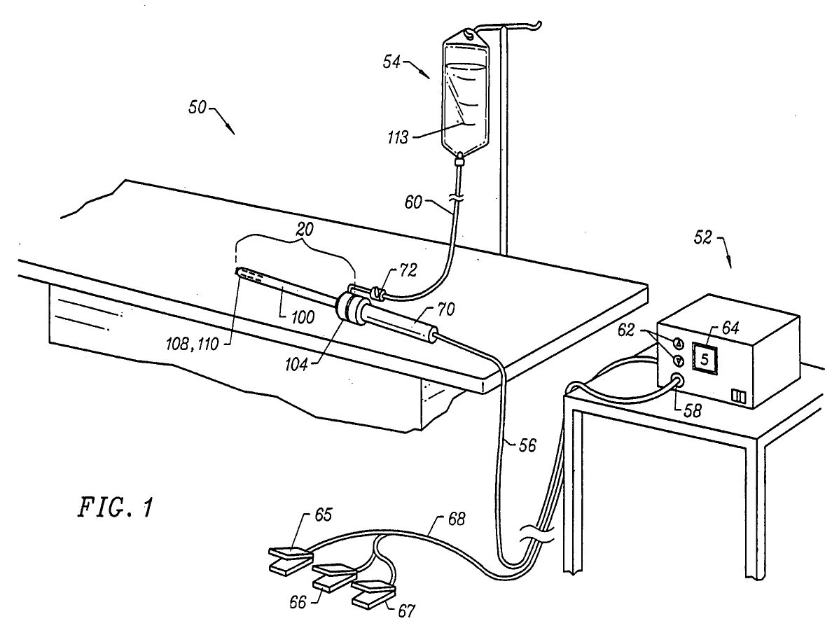

Referring to FIG. 1, an exemplary electrosurgical system 50 for treatment of tissue in the spine will now be described in detail. Electrosurgical system 50 generally comprises an electrosurgical handpiece, handle, or probe 20 connected to a power supply 52 for providing high frequency voltage to a target site, and a fluid source 54 for supplying electrically conductive medium 113 to probe 20. Although the fluid source 54 is illustrated as being directly coupled to the probe 20, the fluid source may be a separate device from the probe. In addition, electrosurgical system 50 may include an endoscope (not shown) with a fiber optic head light for viewing the surgical site. The endoscope may be integral with probe 20, or it may be part of a separate instrument. The system 50 may also include a vacuum source (not shown) for coupling to a suction lumen or tube in the probe 20 for aspirating the target site.

As shown, probe 20 generally includes a proximal handle 104 and an elongate shaft 100 having any number of active electrodes 108, 110 at its distal end. A connecting cable 56 has a connector 58 for electrically coupling the electrodes 108, 110 to power supply 52. The opposite end of the cable 56, contains a hub 70 for connection to the probe 20. The electrodes 108, 110 may be electrically isolated from each other and each electrode is connected to an active or passive control network within power supply 52 by means of a plurality of individually insulated conductors (not shown.) The fluid supply tube 60 is provided for supplying electrically conductive medium 113 to the target site. Although the fluid supply 54 may be driven by gravity, the fluid supply tube 60 may be connected to a suitable pump (not shown), if desired. It is also noted that the system 50 may incorporate a valve member 72 between the fluid supply 54 and the probe 20.

Power supply 52 has an operator controllable voltage level adjustment 62 to change the applied voltage level, which is observable at a voltage level display 64. Power supply 52 also includes first, second and third foot pedals 65, 66, 67 and a cable 68 which is removably coupled to power supply 52. The foot pedals 65, 66, 67 allow the surgeon to remotely adjust the energy level applied to active electrodes 108, 110. In an exemplary embodiment, first foot pedal 65 is used to place the power supply into the “ablation” mode and second foot pedal 66 places power supply 52 into the “sub-ablation” mode (e.g., for coagulation or contraction of tissue.) The third foot pedal 67 allows the user to adjust the voltage level within the “ablation” mode. In the ablation mode, a sufficient voltage is applied to the active electrodes to establish the requisite conditions for molecular dissociation of the tissue (i.e., vaporizing a portion of the electrically conductive medium, ionizing charged particles within the vapor layer and accelerating these charged particles against the tissue.) As discussed above, the requisite voltage level for ablation will vary depending on the number, size, shape and spacing of the electrodes, the distance in which the electrodes extend from the support member, etc. Once the surgeon places the power supply in the “ablation” mode, voltage level adjustment 62 or third foot pedal 67 may be used to adjust the voltage level to adjust the degree or aggressiveness of the ablation.

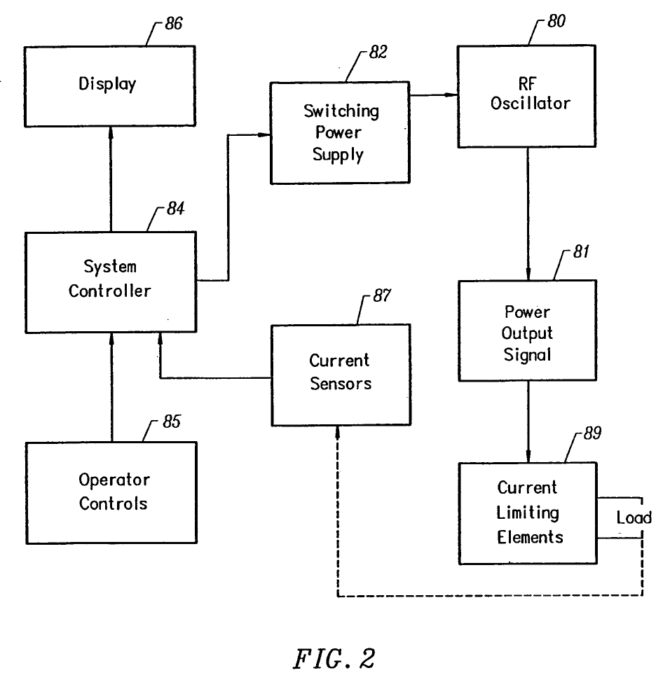

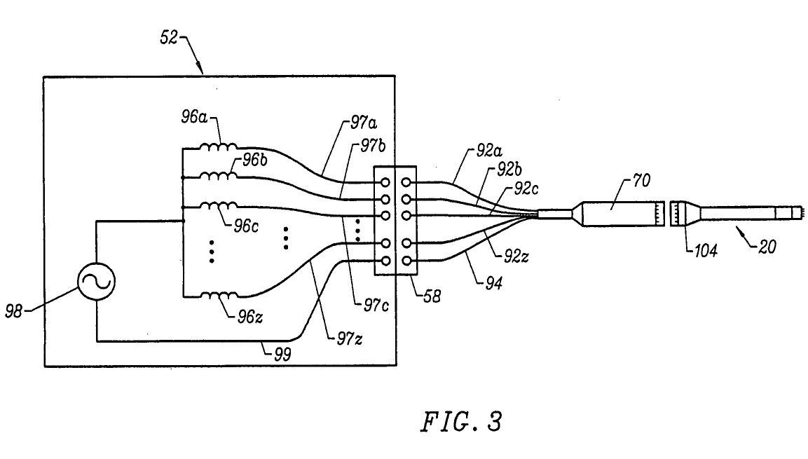

Referring now to FIGS. 2 and 3, a representative high frequency power supply for use according to the principles of the present invention will now be described. The high frequency power supply of the present invention is configured to apply a high frequency voltage of about 10 volts RMS to 500 volts RMS between one or more active electrodes (and/or coagulation electrode) and one or more return electrodes. In the exemplary embodiment, the power supply applies about 70 volts RMS to 350 volts RMS in the ablation mode and about 20 volts to 90 volts in a subablation mode, preferably 45 volts to 70 volts in the subablation mode (these values will, of course, vary depending on the probe configuration attached to the power supply and the desired mode of operation.)

The preferred power source of the present invention delivers a high frequency current selectable to generate average power levels ranging from several milliwatts to tens of watts per electrode, depending on the volume of target tissue being treated, and/or the maximum allowed temperature selected for the probe tip. The power supply allows the user to select the voltage level according to the specific requirements of a particular procedure, e.g., spinal surgery, arthroscopic surgery, dermatological procedure, ophthalmic procedures, open surgery, or other endoscopic surgery procedure.

As shown in FIG. 2, the power supply generally comprises a radio frequency (RF) power oscillator 80 having output connections for coupling via a power output signal 81 to the load impedance, which is represented by the electrode assembly when the electrosurgical probe is in use. In the representative embodiment, the RF oscillator operates at about 100 kHz. The RF oscillator is not limited to this frequency and may operate at frequencies of about 300 kHz to 600 kHz. In particular, for cardiac applications, the RF oscillator will preferably operate in the range of about 400 kHz to about 600 kHz. The RF oscillator will generally supply a square wave signal with a crest factor of about 1 to 2. Of course, this signal may be a sine wave signal or other suitable wave signal depending on the application and other factors, such as the voltage applied, the number and geometry of the electrodes, etc. The power output signal 81 is designed to incur minimal voltage decrease (i.e., sag) under load. This improves the applied voltage to the active electrodes and the return electrode, which improves the rate of volumetric removal (ablation) of tissue.

Power is supplied to RF oscillator 80 by a switching power supply 82 coupled between the power line and the RF oscillator rather than a conventional transformer. The switching power supply 82 allows power supply 82 to achieve high peak power output without the large size and weight of a bulky transformer. The architecture of the switching power supply also has been designed to reduce electromagnetic noise such that U.S. and foreign EMI requirements are met. This architecture comprises a zero voltage switching or crossing, which causes the transistors to turn ON and OFF when the voltage is zero. Therefore, the electromagnetic noise produced by the transistors switching is vastly reduced. In an exemplary embodiment, the switching power supply 82 operates at about 100 kHz.

A controller 84 coupled to the operator controls 85 (i.e., foot pedals and voltage selector) and display 86, is connected to a control input of the switching power supply 82 for adjusting the generator output power by supply voltage variation. The controller 84 may be a microprocessor or an integrated circuit. The power supply may also include one or more current sensors 87 for detecting the output current. The power supply is preferably housed within a metal casing which provides a durable enclosure for the electrical components therein. In addition, the metal casing reduces the electromagnetic noise generated within the power supply because the grounded metal casing functions as a “Faraday shield,” thereby shielding the environment from internal sources of electromagnetic noise.

The power supply generally comprises a main or mother board containing generic electrical components required for many different surgical procedures (e.g., arthroscopy, urology, general surgery, dermatology, neurosurgery, etc.), and a daughter board containing application specific current-limiting circuitry (e.g., inductors, resistors, capacitors and the like.) The daughter board is coupled to the mother board by a detachable multi-pin connector to allow convenient conversion of the power supply to, e.g., applications requiring a different current limiting circuit design. For arthroscopy, for example, the daughter board preferably comprises a plurality of inductors of about 200 to 400 microhenries, usually about 300 microhenries, for each of the channels supplying current to the active electrodes.

Alternatively, in one variation current limiting inductors are placed in series with each independent active electrode, where the inductance of the inductor is in the range of 10 uH to 50,000 uH, depending on the electrical properties of the target tissue, the desired tissue heating rate and the operating frequency. Alternatively, capacitor-inductor (LC) circuit structures may be employed, as described previously in co-pending PCT application No. PCT/US94/05168, the complete disclosure of which is incorporated herein by reference. Additionally, current limiting resistors may be selected. Preferably, these resistors will have a large positive temperature coefficient of resistance so that, as the current level begins to rise for any individual active electrode in contact with a low resistance medium (e.g., saline irrigant or conductive gel), the resistance of the current limiting resistor increases significantly, thereby minimizing the power delivery from said active electrode into the low resistance medium (e.g., saline irrigant or conductive gel.) Power output signal may also be coupled to a plurality of current limiting elements which are preferably located on the daughter board since the current limiting elements may vary depending on the application. A more complete description of a representative power supply can be found in commonly assigned U.S. Pat. No. 6,142,992, previously incorporated herein by reference.

Referring to FIG. 3, a high frequency power supply 52 comprises a voltage source 98 which is connected to a multiplicity of current limiting elements 96a, 96b, . . . , 96z, typically being inductors having an inductance in the range from 100 to 5000 microhenries, with the particular value depending on the electrode terminal dimensions, the desired ablation rates, and the like. In the case of ablation of articular and fibrocartilage, suitable inductances will usually be in the range from 50 to 5000 microhenries. Capacitors having capacitance values in the range from 200 to 10,000 picofarads may also be used as the current limiting elements.

Current limiting elements may also be part of a resonant circuit structure having a capacitor in series with the electrode terminal and an inductor between the electrode lead and the common lead. The inductor and capacitor values are selected according to the operating frequency of the voltage source 98. By way of example, at an operating frequency of 100 kHz, current limiting circuit structures may incorporate inductor/capacitor combinations such as (1) 2530 microhenries and 1000 picofarads; (2) 5390 microhenries and 470 picofarads; or (3) 11,400 microhenries and 220 picofarads, respectively.

It would also be possible to use resistors as the current limiting elements. The use of resistors, however, is generally less preferred than use of inductors or capacitor/inductor tuned circuit structures since resistors will have significant IR² power losses which are generally avoided with the circuits of FIG. 3.

Referring to FIG. 3, each of the individual leads 97 from the current limiting elements 96 are removably connected to leads 92 via connector 58. A common electrode lead 99 from voltage source 98 is removably connected to lead 94 via the same connector 58. Each of the electrode leads 92 and common electrode lead 94 extend into and through handle 70 and terminate the distal end of handle 70. As described with reference to FIG. 3, electrical leads 92 and common electrode lead 94 connect to electrodes in the probe 20. In this manner, each of the electrodes in the device 20 can be powered by a single voltage source 98 with independent current limiting elements or circuit structures attached to each electrode via cable lead 92 and controller lead 96.

Current limitation could alternatively be accomplished by providing a separate power supply and current measuring circuitry for each electrode terminal. Current flow to any electrode terminal which exceeds a predetermined (or adjustable) limit would be decreased or interrupted.

The following illustrations are examples of the invention described herein. It is contemplated that combinations of aspects of specific embodiments or combinations of the specific embodiments themselves are within the scope of this disclosure.

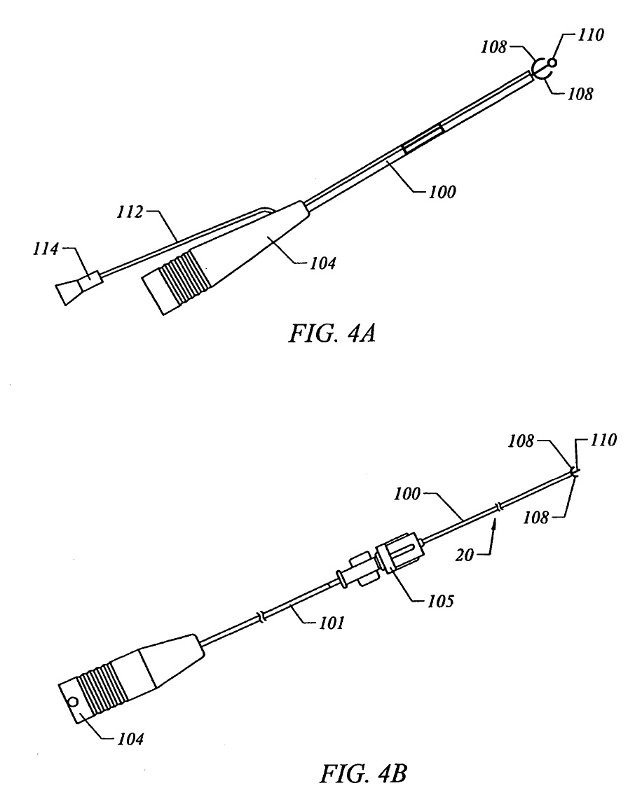

FIG. 4 illustrates a variation of an inventive electrosurgical probe 20 of the present invention. As shown in FIG. 4, probe 20 generally includes an elongated shaft 100 which may be flexible or rigid, a handle 104 coupled to the proximal end of shaft 100. The electrodes 108, 110 may be coupled to an electrode support member (not shown) located at the distal end of shaft 100. Some variations of the device may have a shaft 100 which comprises an electrically conducting material, usually metal, which may be selected from the group comprising tungsten, stainless steel alloys, platinum or its alloys, titanium or its alloys, molybdenum or its alloys, and nickel or its alloys. In one variation, the shaft 100 includes an electrically insulating jacket, which is typically formed as one or more electrically insulating sheaths or coatings, such as polytetrafluoroethylene, polyimide, and the like. The electrically insulating jacket over the shaft prevents direct electrical contact between these metal elements and any adjacent body structure or the surgeon. Such direct electrical contact between a body structure (e.g., tissue, vertebral disc, tendon, cartilage, blood, other bodily fluids, etc.) and an exposed electrode could result in unwanted heating and necrosis of the structure at the point of contact. It is also contemplated that the shaft 100 may be fabricated from a non-conductive material.

The shaft preferably has a length in the range of about 4 to 30 cm. In one aspect of the invention, probe is manufactured in a range of sizes having different lengths and/or diameters of shafts. A shaft of appropriate size can then be selected by the surgeon according to the body structure or tissue to be treated and the age or size of the patient. In this way, patients varying in size from small children to large adults can be accommodated. Similarly, for a patient of a given size, a shaft of appropriate length and diameter can be selected by the surgeon depending on the organ or tissue to be treated, for example, whether an intervertebral disc to be treated is in the lumbar spine or the cervical spine. For example, a shaft suitable for treatment of a disc of the cervical spine may be substantially smaller than a shaft for treatment of a lumbar disc. For treatment of a lumbar disc in an adult, the shaft length is preferably in the range of about 15 to 25 cm. For treatment of a cervical disc, the shaft 100 length is preferably in the range of about 4 to about 15 cm. In some cases, introducer needles used for the treatment of lumbar discs range from 14 Gauge to 22 Gauge.

The present invention may also incorporate depth markings to provide information on how far the probe has been advanced. For example, in certain variations, depth markings may be present along the entire length of the probe, or a single depth marking may be present at the shaft proximal end portion. Depth markings serve to indicate to the surgeon the depth of penetration of shaft into a patient's tissue, organ, or body during a surgical procedure. Depth markings may be formed directly in or on the probe. The depth markings may be incorporated on the probe through a number of conventional methods, including, but not limited to laser printing, etching, and/or pad printing, etc. The depth markings may be formed from materials which have a different color and/or a different level of radiopacity, as compared with material of probe. For example, depth markings may comprise a metal, such as tungsten, gold, or platinum oxide (black), having a level of radiopacity different from that of adjacent portions of the probe. Such depth markings may be visualized by the surgeon during a procedure performed under fluoroscopy.

The handle 104 may comprise a plastic material that is easily molded into a suitable shape for handling by the surgeon. Handle 104 defines an inner cavity (not shown) that houses the electrical connections (as described herein), and provides a suitable interface for connection to an electrical connecting cable distal portion. Although not illustrated, the handle 104 may be integrated with a cable that connects to a power supply.

The probe 20 also includes electrodes 108, 110 located at a distal end of the shaft 100. The electrodes 108, 110 may be placed in an electrode support member (not shown) which is either flush with the distal end of the shaft 100 or extends from the distal end usually about 1 mm to 20 mm. The electrode support member provides support for any number of the electrodes 108, 110.

As shown in FIG. 4A, a variation of the invention may include a fluid delivery tube 112 that extends along the device 20. It should be noted that although the fluid delivery tube 112 is depicted as being external to the shaft 100, the fluid delivery tube 112 may be incorporated within the shaft 100. Moreover, a fluid delivery tube may be provided separate from the device 20. The fluid delivery tube 112 may contain one or more lumens that extend through shaft 100 to one or more openings at its distal end. In any event, the fluid delivery tube 112 is ultimately fluidly coupled to a connector 114 enabling the fluid delivery tube 112 to be coupled to a fluid supply source. The probe 20 may also include a valve (not shown) or equivalent structure for controlling the flow rate of the electrically conductive medium to the target site. As described herein, it is desirable to provide a supply of electrically conductive medium to the target site.

As shown in the variation depicted in FIG. 4A, the probe 20 includes at least one active electrode 108 and a return electrode 110. The active and return electrodes 108, 110 are coupled to opposite poles of the power supply and form part of the current path between the poles of the power supply. A more detailed explanation of the electrode assembly follows below.

FIG. 4B illustrates another variation of the inventive device 20. In this variation the probe 20 does not include a fluid delivery tube. Accordingly, this variation of the probe 20 is suitable for use in a fluid filled environment or one where bodily fluids provide a conductive path between electrodes 108, 110. As illustrated, the distal end of the shaft 100 includes electrodes 108, 110. The proximal end of the shaft 100 may include one or more additional handles or hubs 105. The hub 105 may connect to an extension cable or “pig-tail” 101 which is ultimately connected to a handle 104 that is adapted to couple to the power supply (the handle 104 may be connected to an additional cable, or may have an integral cable for coupling to the power supply.) The hub 105 may be a separate catheter, such as a commonly known break-away-introducer which assists the medical practitioner in placing the device. Or, the hub 105 may be affixed to the shaft 100 and/or pig-tail 101 thereby assisting the medical practitioner in positioning of the device. Furthermore, the hub 105 may incorporate features for use in determining the travel distance within the body (e.g., functioning as a stop-mechanism.)

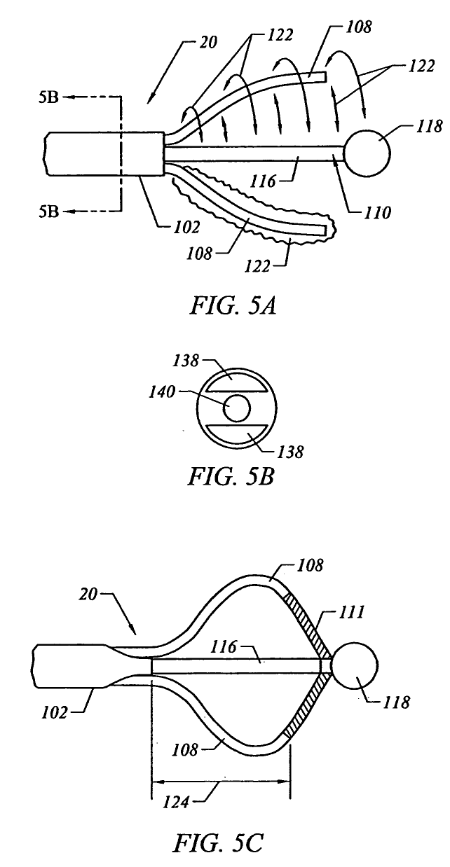

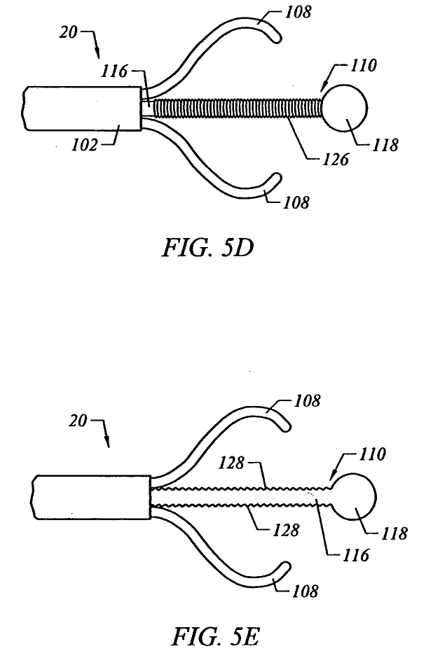

FIGS. 5A-9B illustrate variations of the distal end of a probe of the invention. FIG. 5A illustrates a basic variation of a working end of a probe 20 of the invention. The electrodes 108, 110 may be attached to a support structure 102. The support structure 102 may be located in the distal end of the shaft 100. Preferably, the support structure is a non-conductive material. The preferred support matrix material is alumina, available from Kyocera Industrial Ceramics Corporation, Elkgrove, Ill., because of its high thermal conductivity, good electrically insulative properties, high flexural modulus, resistance to carbon tracking, biocompatibility, and high melting point. In one variation, the support matrix 102 is adhesively joined to a tubular support member (not shown) that extends most or all of the distance between matrix 102 and the proximal end of probe. Tubular member preferably comprises an electrically insulating material, such as an epoxy or silicone-based material.

It should be noted that for some variations of the invention, the electrodes may be placed directly into the shaft 100. (e.g., from FIG. 5A, the shaft would be depicted by 102) The shaft may have one or more lumens, but in most variations it is desirable to individually insulate the active and return electrodes. For example, as shown in FIG. 5B, the support 102 may comprise a multi-lumen shaft, in which case the shaft 102 will have separate lumens 138 for the active electrodes and a lumen 140 for the return electrode.

The electrodes are configured to assume the expanded profile illustrated in FIG. 5A. Accordingly, restraining the electrodes in a reduced profile, (e.g., moving the active electrodes 108 such that they are immediately adjacent to the return electrode 110) allows the working end of the device to be introduced into the body via a smaller diameter opening. The electrodes, especially, the active electrodes 108, may be fabricated from a resilient spring material or a shape memory alloy such that they assume the expanded profile once any restraint is released from the reduced profile. The active 108 and return 110 electrodes may be constructed from titanium, tantalum, steel, stainless steel, tungsten, copper, gold or the like. In some variations, an electrode may be formed using a base material having the desired mechanical properties (i.e., modulus of elasticity, shape memory alloy, etc.) that is coated with a desirable conductive material.

It should be noted, that in the variations of the probe discussed herein, the support structure 102 and electrodes 108, 110 may be slidably located within a shaft. Accordingly, when the electrodes 108, 110 are placed within the shaft they assume the reduced profile. Alternatively, they may be affixed to the shaft. In the latter case, an outer sheath (not illustrated) may be used to restrain the electrodes 108, 110 in the reduced profile. Upon release of the electrodes from any restraint (e.g., the shaft, an outer sheath, peel-away introducer, etc.), the electrodes assume the expanded profile via, e.g., elastic return, shape memory effect, etc.

Turning now to the electrode configuration, it is understood that the active electrode(s) 108 and the return electrode 110 are coupled to opposite poles of a power supply. Any number of redundant joint configurations as known by those skilled in the art may be used to affix the electrodes to the device. The return electrode 110 comprises a return body portion 116 and a tip portion 118. Although two active electrodes 108 are illustrated, the invention contemplates variations of the device as having one or more active electrodes 108. Furthermore, any portion of the electrodes may be covered with an insulating layer (not illustrated.) The remaining uninsulated conducting portion of the electrode would function to form a portion of the current path. It is important to note that the surface area of the return electrode 110 should be greater than the surface area of the active electrodes 108. This configuration properly allows an ablative effect to occur at the active 108 rather than the return 110 electrodes. Furthermore, the tip portion 118 of the return electrode 110 will extend distally of the active electrode 108. Accordingly, since the ablation effect primarily takes place at the active electrode 108 rather than the return electrode 110, the tip portion 118 of the return electrode 110 may be used as a mechanical stop/limit when using the device to ablate tissue. It should be noted that a mechanical stop may also be included on the shaft of the device. However, it is important that the tip portion 118 of the return electrode 110 does not contain sharpened edges such that current density increases at the return electrode 110.

As depicted in FIG. 5A, the lines 122 between the active 108 and return 110 electrode are intended to illustrate current flux between the electrodes. (For clarity, the current flux 122 is only depicted between the top active electrode 108 and the return electrode 110.) Given the surface area differential between active and return electrodes, as described above, a high current density will develop adjacent to the active electrode 108 thereby forming a plasma layer around the active electrode 108 (in the presence of an electrically conductive medium and as illustrated about the lower active electrode 108.) As discussed herein, it is believed that the plasma layer drives ablation of tissue in the region adjacent to the active electrode 108.

As shown in FIG. 5A, return electrode 110 is not directly connected to active electrodes 108. To complete this current path so that active electrodes 108 are electrically connected to return electrode 110, an electrically conductive medium (e.g., isotonic saline) is caused to flow therebetween. In the illustration shown in FIG. 4A, the electrically conductive medium is delivered through fluid tube 112 to an opening in the distal end of the probe 20. Alternatively, the conductive medium may be delivered by a fluid delivery element (not shown) that is separate from probe 20. In arthroscopic surgery, for example, the target area of the joint will be flooded with isotonic saline and the probe 20 will be introduced into this flooded target area. Electrically conductive medium can be continually resupplied to maintain the conduction path between return electrode 110 and active electrodes 108. In other embodiments, the distal portion of probe 20 may be dipped into a source of electrically conductive medium, such as a gel or isotonic saline, prior to positioning at the target site. Applicant has found that the surface tension of the fluid and/or the viscous nature of a gel allows the conductive medium to remain around the active and return electrodes for long enough to complete its function according to the present invention, as described below. Alternatively, the conductive medium, such as a gel, may be applied directly to the target site (e.g., injected into the site prior to the procedure). Furthermore, the composition of some portions of the body may have a naturally occurring medium (e.g., a vertebral disc which has a high saline content.) When used in such areas, an extraneous conductive medium is not required.

Variations of the invention include probes having a fluid path that is formed in probe 20 by, for example, an inner lumen or an annular gap between the return electrode 110 and a tubular support member within shaft 100. This annular gap may be formed near the perimeter of the shaft 100 such that the electrically conductive medium tends to flow radially inward towards the target site, or it may be formed towards the center of shaft 100 so that the fluid flows radially outward. In both of these variations, a fluid source (e.g., a bag of fluid elevated above the surgical site or having a pumping device) is coupled to probe 20 via a fluid supply tube (not shown) that may or may not have a controllable valve. A more complete description of an electrosurgical probe incorporating one or more fluid lumen(s) can be found in U.S. Pat. No. 5,697,281, the complete disclosure of which has previously been incorporated herein by reference.

FIG. 5C illustrates another variation of a distal end of the invention. As shown, the active electrode 108 may be shaped to produce a more desirable current path or it may have a shape that promotes advancement of the device within tissue, aids in delivering the device through the introducer needle, and/or smooth retraction back into the access cannula at the end of the procedure. The arm portion span 124 is measured as shown on FIG. 5C. It was found that shorter arms increased ratio of the return electrode surface area to the active electrode surface area. As discussed above, increasing this ratio allows for increased current density along the arm portion of the active electrodes 108 thereby allowing formation of a plasma layer around the active electrodes 108. Moreover, a shorter portion span 124 increases the durability of the arms as there is less of a chance of bending of the arms as the device is advanced through tissue (in cases where the return electrode 124 has a free end). It was found that the arm portion span 124 may be greater than 1 mm.

In an additional variation, and as illustrated in FIG. 5C, at a distal end of the shaft 102 the walls of the shaft 102 may be removed to allow the active electrodes 108 to diverge from the return electrode. However, it is important that the wall of the return electrode lumen 140 remain so that there is no direct contact between the electrodes. As mentioned herein, the cross-sectional configuration of the shaft 102 may vary to accommodate any number of electrodes. It was found that an acceptable shaft 102 material included, for example, PTFE or ETFE.

FIG. 5C also illustrates another variation of the invention where the active electrode 108 includes an electrode support 111. In such a case, the return electrode 110 may be moveable relative to the active electrodes 108 to allow for the active electrodes to assume an expanded or reduced profile. The electrode support 111 provides an additional safeguard to prevent the active electrodes 108 from breaking off of the device during use. It should be noted that the electrode support 111 will be insulated from the return electrode so that current cannot flow directly therebetween. Furthermore, in a simple variation, the electrode support 111 may be comprised of the same material as the active electrode 108 with an insulating layer placed over the support portion 111. It is also noted that the return electrode 108 may be located distally to the electrode support 111, in such a case the proximal portion of the electrode 108 will be insulated to serve as the electrode support 111. Furthermore, the majority of the electrode 108 may be conductive with just a small portion serving as the support 111. In such a case, the support 111 would insulate the active electrode 108 from the return electrode.

FIG. 5D illustrates another variation of the invention. In this variation, the probe 20 comprises an electrode assembly having active 108 and return 110 electrodes where the return electrode 110 comprises a conductive material 126 coiled around the body portion 116 of the return 110 electrode. This conductive material 126 may be a coiled wire or other member. The purpose of the conductive material 126 is to increase the surface area of the return electrode 110. As discussed herein, increasing the return electrode surface area produces a desirable current path and current density around the active electrodes 108. The conductive material 126 may be fabricated from the same or from different material as the electrodes. Also, the conductive material 126 may have a fine pitch (as illustrated) or may have a coarse pitch (e.g., leaving spaces between the coils of the material to expose the body portion 116 of the return electrode 110.)

FIG. 5E illustrates another variation of the invention similar to that of FIG. 5D. In this variation, the body portion 116 of the return electrode 110 contains segments of raised surfaces 128. As discussed above, the raised surfaces 128 increase the ratio of the return electrode surface area to the active electrode surface area. The raised surfaces 128 may be created in the arm portion 116 via etching, machining, EDM, etc. However, the raised surfaces 128 should not have sharp edges or be of a size small enough to increase the current density at the return electrode 110.

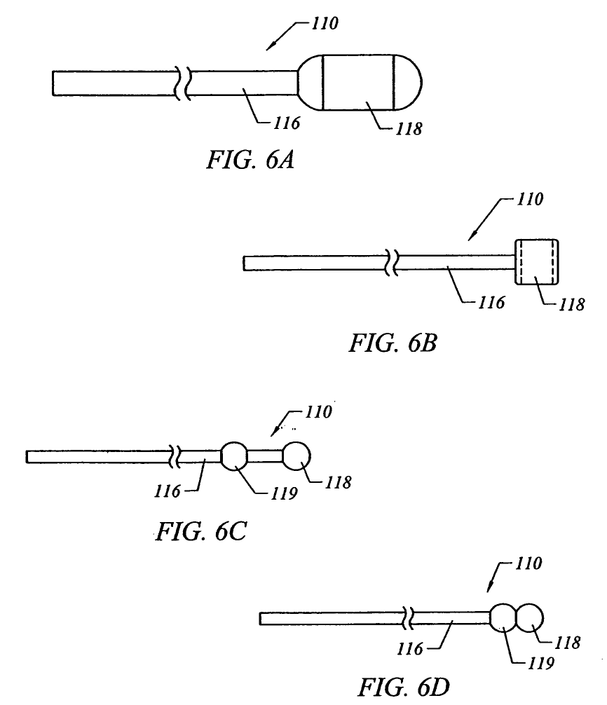

FIGS. 6A-6D illustrate additional variations of return electrodes 110 for use with any of the devices of the present invention. In each variation, the return electrode 110 contains a sufficiently large surface area to produce a desirable current path and current density around the active electrodes (not shown in FIGS. 6A-6D.) The blunt structure of the return electrodes 110 also permits the return electrode 110 to function as a “bumper” when advanced into an inner side of an annulus of a vertebral disc. Accordingly, when the device is advanced into the annulus wall it will not penetrate or compromise the annulus because the return electrode 110 is configured to form the ablation layer at the proximally located active electrodes and because the return electrode 110 is atraumatic or blunt.

FIG. 6A illustrates an ‘elongated’ return electrode 110 having a distal tip portion 118 along with a body portion 116. Either the entirety or a portion of the distal tip 118 may be conductive. Moreover, the body portion 116 may or may not be conductive as well. However, the tip portion 118 shown in this figure is atraumatic and has rounded edges. These features reduce the ability of the device to form an ablation zone at the distal tip 118 and further reduce the possibility that the distal tip 118 of the device will compromise the annulus of a disc.

FIG. 6B illustrates another variation of a return electrode 110 having a distal tip portion 118 and a body portion 116. For reasons discussed herein, the edges of the distal tip 118 will be rounded to produce a desirable current density and to provide an atraumatic tip.

FIGS. 6C-6D illustrate another variation of a return electrode for use with the inventive device. In these variations, the return electrode 110 further includes a return portion 119. The return portion 119 is conductive and provides an increased surface area for reasons described herein. The tip portion 118 of these variations may or may not be conductive. In the latter case, the tip portion 118 serves solely as a ‘bumper’ and atraumatic tip. As shown in FIGS. 6C and 6D, the return portion 119 may or may not be separated from the tip portion 118. Although not depicted, in one variation of the device, the active electrodes are proximate to the return portion but the inventive device is not limited to such a configuration.

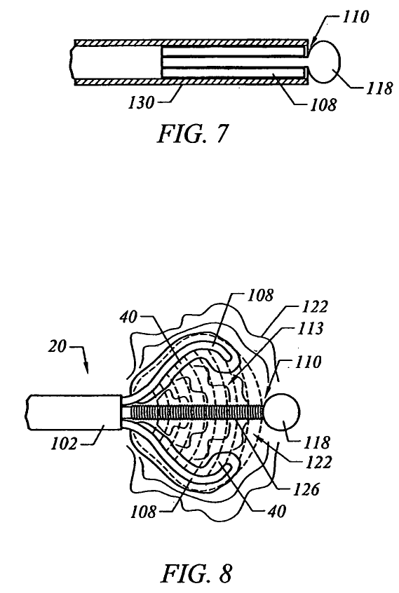

FIG. 7 illustrates a variation of the inventive device having of an outer sheath or covering 130 located about the shaft and electrodes 108, 110. As illustrated, as the sheath 130 slides over the active electrodes 108, the electrodes 108 assume a reduced profile. FIG. 7 also illustrates an additional variation of the distal tip portion 118 of the return electrode 110. As shown, the tip portion 118 of the return electrode 110 is not limited to being spherical. For example, the tip portion 118 may comprise a shape such as a semi-sphere, an oblate sphere, a prolate sphere, a rounded triangular shape, etc. However, it is important that the tip portion 118 of the return electrode 110 does not contain sharpened edges such that current density increases at the return electrode 110.

As shown in FIG. 8, in the inventive device, the return electrode 110 is not directly connected to active electrodes 108. To complete this current path so that electrodes 108 are electrically connected to return electrode 110, electrically conducting liquid 113 (e.g., isotonic saline) is placed between the electrodes 108, 110. As discussed above, the fluid may be supplied by a lumen incorporated in the probe 20, by an external electrically conductive medium source, or by the fluid already present in the operative site. When a voltage difference is applied between active electrodes 108 and return electrode 110, high electric field intensities will be generated along the active electrodes 108 due to the current flux lines 122 creating an increased current density adjacent thereto. The high electric field intensities cause ablation of tissue, disc material, and/or nucleus material, in regions 40. In this manner, an ablation zone is created as the device is advanced and/or rotated through the body structure.

In some applications, this current flow path 122 results in a deeper current penetration into the surrounding tissue with the same voltage level, and thus increased thermal heating of the tissue. As discussed above, this increased thermal heating may have advantages in some applications of treating disc or other spinal abnormalities. Typically, it is desired to achieve a tissue temperature in the range of about 60° C. to 100° C. to a depth of about 0.2 mm to 5 mm, usually about 1 mm to 2 mm. The voltage required for this thermal damage will partly depend on the electrode configurations, the conductivity of the tissue and the area immediately surrounding the electrodes, the time period in which the voltage is applied and the depth of tissue damage desired. With the electrode configurations described herein, the voltage level for thermal heating will usually be in the range of about 20 volts rms to 300 volts rms, preferably about 60 volts rms to 200 volts rms. The peak-to-peak voltages for thermal heating with a square wave form having a crest factor of about 2 are typically in the range of about 40 to 600 volts peak-to-peak, preferably about 120 to 400 volts peak-to-peak. The higher the voltage is within this range, the less time required. If the voltage is too high, however, the surface tissue may be vaporized, debulked or ablated, which is undesirable.

It is noted that the devices described herein are able to perform in a thermal heating mode (coagulation) alone, or in conjunction with an ablation mode. In the thermal heating mode a lower voltage is typically applied below the threshold for plasma formation and ablation, but sufficient to cause some thermal damage to the tissue immediately surrounding the electrodes without vaporizing or otherwise debulking this tissue so that the current provides thermal heating and/or coagulation of tissue surrounding electrodes.

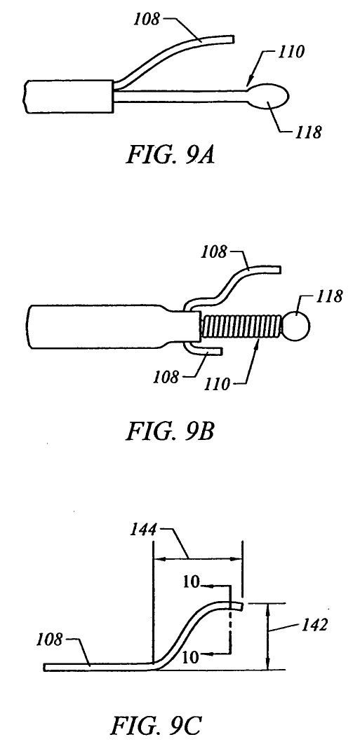

FIGS. 9A-9B illustrate additional configurations of the active electrode 108 of the present invention. As illustrated in FIG. 9A, the active electrode 108 may be a single electrode located about the return electrode 110. Alternatively, the active electrode 108 may comprise any number of electrodes spaced about the return electrode 110 so long as the ratio of the return electrode surface area to the active electrode surface area remains sufficiently high.

FIG. 9B illustrates an example of the inventive device having active electrodes 108 of different lengths. Such a variation may be used to ensure ablation along the entire length of the longer active electrode 108.

FIG. 9C illustrates a basic example of an active electrode 108 of the present invention. The height 142 and length 144 of the active electrode are measured as illustrated. It is believed that proper selection of these dimensions increases the probability that the active electrode 108 will form a desirable ablation field.

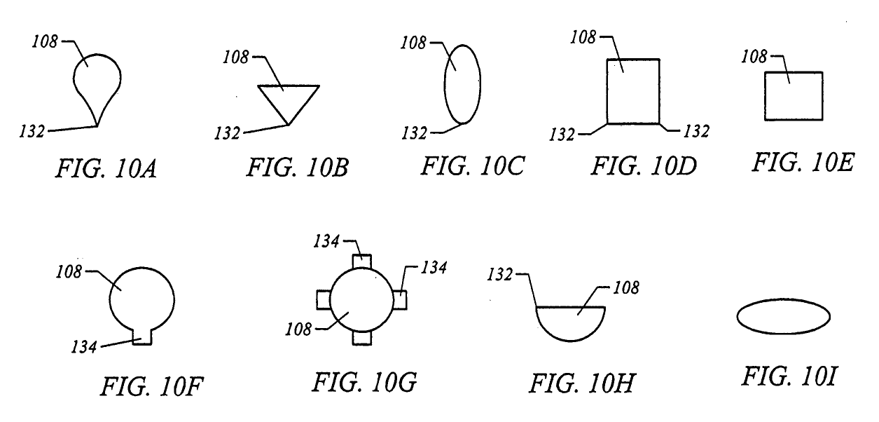

FIGS. 10A-10I provide a sample of the variations of cross section of the active electrode as taken along the line 10-10 in FIG. 9C. The cross sectional shape of the active electrode 108 may include d-shape, square, rectangular, triangular, circular, oval, etc. For example, the active electrode 108 may be a flattened wire having a rectangular cross sectional shape. In each case the active electrode 108 may have a section of reduced surface area as denoted by 132. As discussed herein, the reduced surface area promotes current density along the electrode. Furthermore, as shown in FIGS. 10F and 10G, basic geometric shapes may be combined with keyways or protrusions 134 to provide additional sections of reduced surface area. The sections of reduced surface area 132, 134 may extend along a portion, or the entire active electrode 108. The cross sectional shapes discussed above are merely examples of shapes for the electrode. Additional shapes may be selected from commercially available stock suppliers such as Fort Wayne Metal Research Products Corporation.

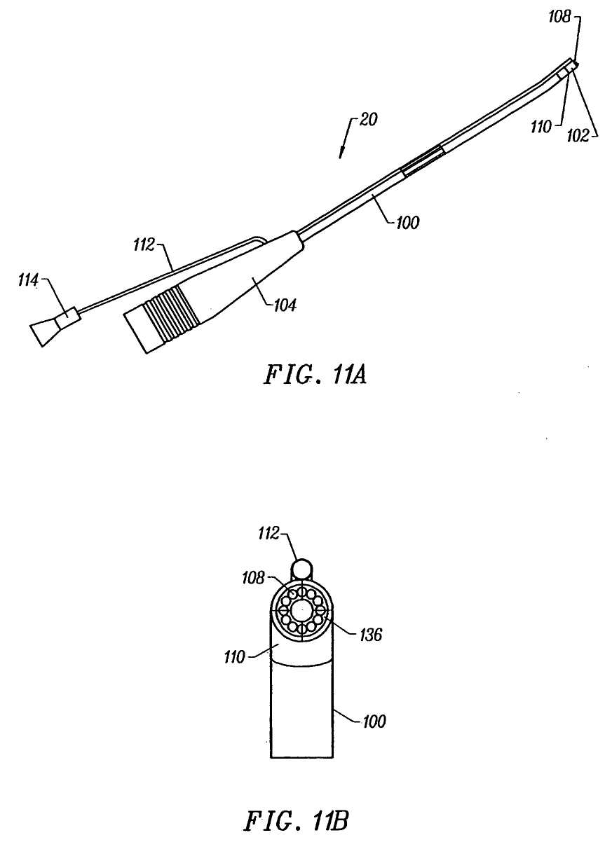

FIGS. 11A-11B illustrate additional variations of electrosurgical probes 20 for use with the inventive procedure described herein. As shown in FIG. 11A, probe 20 generally includes an elongated shaft 100 which may be flexible or rigid, a handle 104 coupled to the proximal end of shaft 100 and an electrode support member 102 coupled to the distal end of shaft 100. Shaft 100 preferably comprises an electrically conducting material, usually metal, which may be selected from the group comprising tungsten, stainless steel alloys, platinum or its alloys, titanium or its alloys, molybdenum or its alloys, and nickel or its alloys. In one variation, the shaft 100 includes an electrically insulating jacket (not shown), which is typically formed as one or more electrically insulating sheaths or coatings, such as polytetrafluoroethylene, polyimide, and the like. The electrically insulating jacket over the shaft prevents direct electrical contact between these metal elements and any adjacent body structure or the surgeon.

Electrode support member 102 extends from the distal end of shaft 100 (usually about 1 mm to 20 mm), and provides support for any number of electrically isolated active electrodes 108. As shown in FIG. 11A, a variation of the invention may include a fluid tube 112 that extends through an opening in handle 104, and includes a connector 114 for connection to a fluid supply source, for supplying electrically conductive medium to the target site. Depending on the configuration of the distal surface of shaft 100, fluid tube 112 may extend through a single lumen (not shown) in shaft 100, or it may be coupled to a plurality of lumens (also not shown) that extend through shaft 100 to a plurality of openings at its distal end. In the representative embodiment, tubing 112 is a tube that extends along the exterior of shaft 100 to a point just distal of return electrode 110. In this embodiment, the fluid is directed through an opening past return electrode 110 to the active electrodes 108. Probe 20 may also include a valve or equivalent structure for controlling the flow rate of the electrically conductive medium to the target site.

As shown in FIG. 11A, variations of the device may have a distal portion of shaft 100 that is bent to improve access to the operative site. In this variation, electrode support member 102 has a substantially planar tissue treatment surface (FIG. 11B) that is usually at an angle of about 10 degrees to 90 degrees relative to the longitudinal axis of shaft 100, preferably about 30 degrees to 60 degrees and more preferably about 45 degrees. In alternative embodiments, the distal portion of shaft 100 comprises a flexible material which can be deflected relative to the longitudinal axis of the shaft as described above.

In the variations shown in FIGS. 1A-11B, probe 20 includes a return electrode 110 for completing the current path between active electrodes 108 and a high frequency power supply. As shown, return electrode 110 preferably comprises an exposed portion of shaft 100 shaped as an annular conductive band near the distal end of shaft 100 slightly proximal to tissue treatment surface of electrode support member 102, typically about 0.5 mm to 10 mm and more preferably about 1 mm to 10 mm. Return electrode 110 or shaft 100 is coupled to a connector that extends to the proximal end of probe 20, where it is suitably connected to power supply (e.g. see FIG. 1.) Return electrode 110 is not directly connected to active electrodes 108. As discussed above, an electrically conductive medium completes this current path so that active electrodes 108 are electrically connected to return electrode 110.

Referring to FIG. 11B, the electrically isolated active electrodes 108 are spaced apart over tissue treatment surface of electrode support member 102. The tissue treatment surface 136 and individual active electrodes 108 will usually have dimensions within the ranges set forth above. In the representative embodiment, the tissue treatment surface 136 has a circular cross-sectional shape with a diameter in the range of 1 mm to 20 mm. The individual active electrodes 108 preferably extend outward from tissue treatment surface 136 by a distance of about 0.1 mm to 4 mm, usually about 0.2 mm to 2 mm. Applicant has found that this configuration increases the high electric field intensities and associated current densities around active electrodes 108 to facilitate the ablation and shrinkage of tissue as described in detail above.

A more detailed discussion of alternate variations of devices for use with the inventive method are included in commonly assigned U.S. Pat. No. 5,697,281 the complete disclosure of which was previously incorporated by reference, and pending U.S. application Ser. Nos. 09/676,194; 09/747,311; 09/679,394; 60/299,095; and 60/322,015 the complete disclosures of which are incorporated herein by reference.

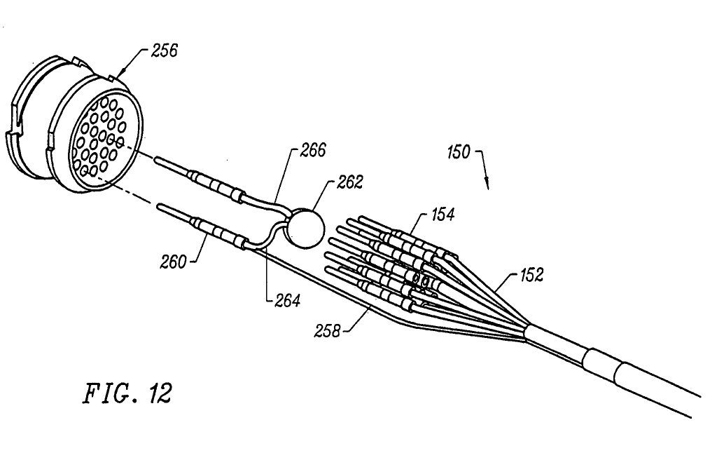

FIG. 12 illustrates an example of electrical connections 150 within handle 104 for coupling active electrode(s) and return electrode to the power supply. As shown, a plurality of wires 152 extend through shaft to couple active electrodes to a plurality of pins 154, which are plugged into a connector block 256 for coupling to a connecting cable distal end (see e.g., FIG. 1.) Similarly, return electrode is coupled to connector block 256 via a wire 258 and a plug 260.

According to the present invention, the probe 20 further includes an identification element 262 that is characteristic of the particular electrode assembly so that the same power supply can be used for different electrosurgical operations. In one embodiment, for example, the probe (e.g., 20) includes a voltage reduction element or a voltage reduction circuit for reducing the voltage applied between the active electrodes and the return electrode. The voltage reduction element serves to reduce the voltage applied by the power supply so that the voltage between the active electrodes and the return electrode is low enough to avoid excessive power dissipation into the electrically conducting medium and/or ablation of the soft tissue at the target site. In some embodiments, the voltage reduction element allows the power supply to apply two different voltages simultaneously to two different electrodes.

In other variations, the voltage reduction element primarily allows the electrosurgical probe to be compatible with various electrosurgical generators supplied by ArthroCare Corporation (Sunnyvale, Calif.) that are adapted to apply higher voltages for ablation or vaporization of tissue. For thermal heating or coagulation of tissue, for example, the voltage reduction element will serve to reduce a voltage of about 100 volts rms to 170 volts rms (which is a setting of 1 or 2 on the ArthroCare Model 970 and 980 (i.e., 2000) Generators) to about 45 volts rms to 60 volts rms, which is a suitable voltage for coagulation of tissue without ablation (e.g., molecular dissociation) of the tissue.

Of course, for some procedures, the probe will typically not require a voltage reduction element. Alternatively, the probe may include a voltage increasing element or circuit, if desired. Alternatively or additionally, the cable and/or cable distal end that couples the power supply to the probe may be used as a voltage reduction element. The cable has an inherent capacitance that can be used to reduce the power supply voltage if the cable is placed into the electrical circuit between the power supply, the active electrodes and the return electrode. In this embodiment, the cable distal end may be used alone, or in combination with one of the voltage reduction elements discussed above, e.g., a capacitor. Further, it should be noted that the present invention can be used with a power supply that is adapted to apply a voltage within the selected range for treatment of tissue. In this embodiment, a voltage reduction element or circuitry may not be desired.

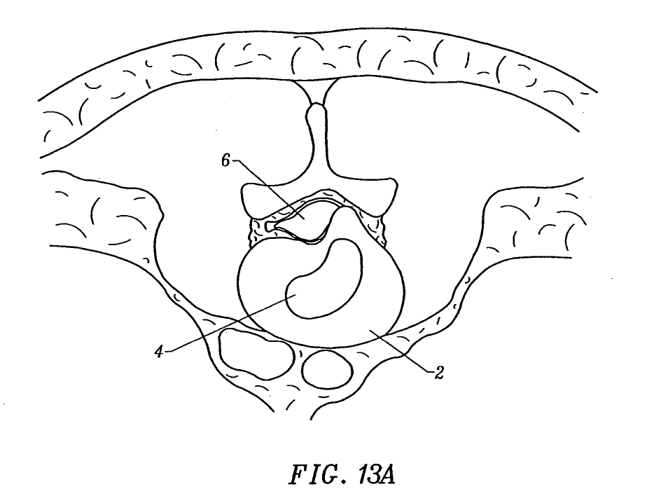

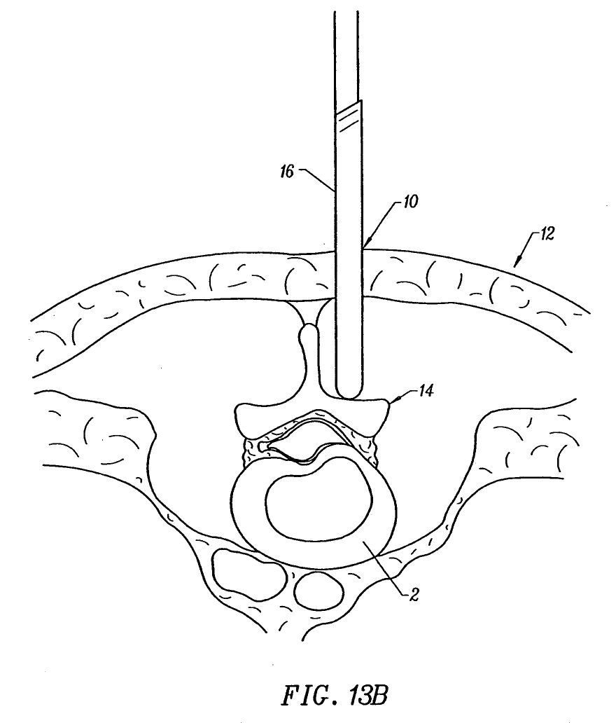

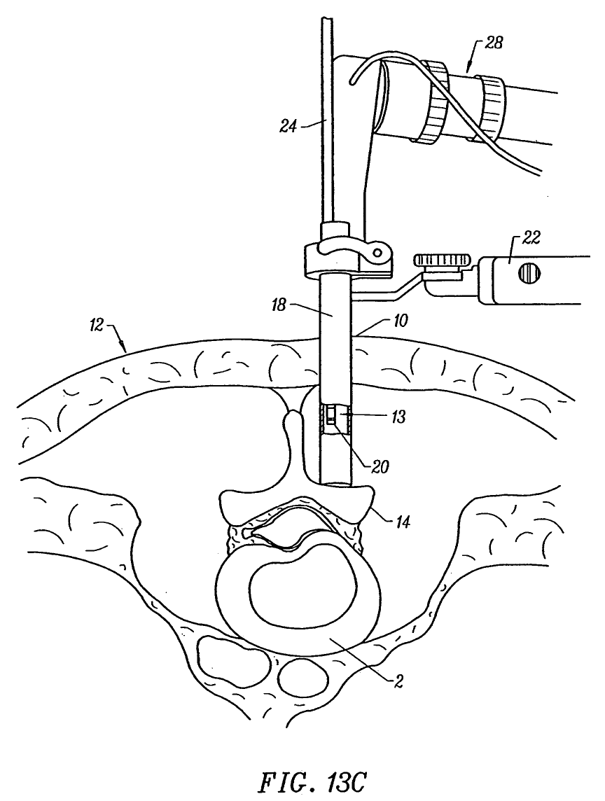

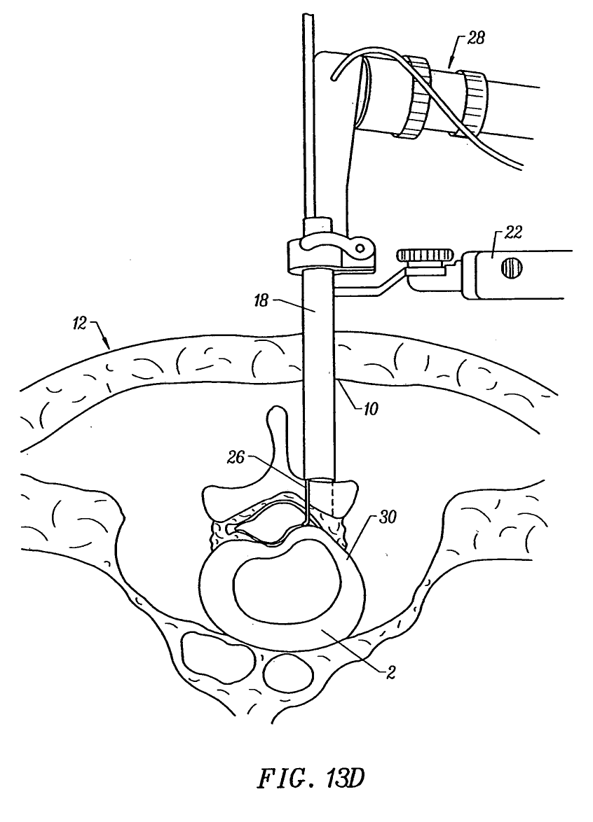

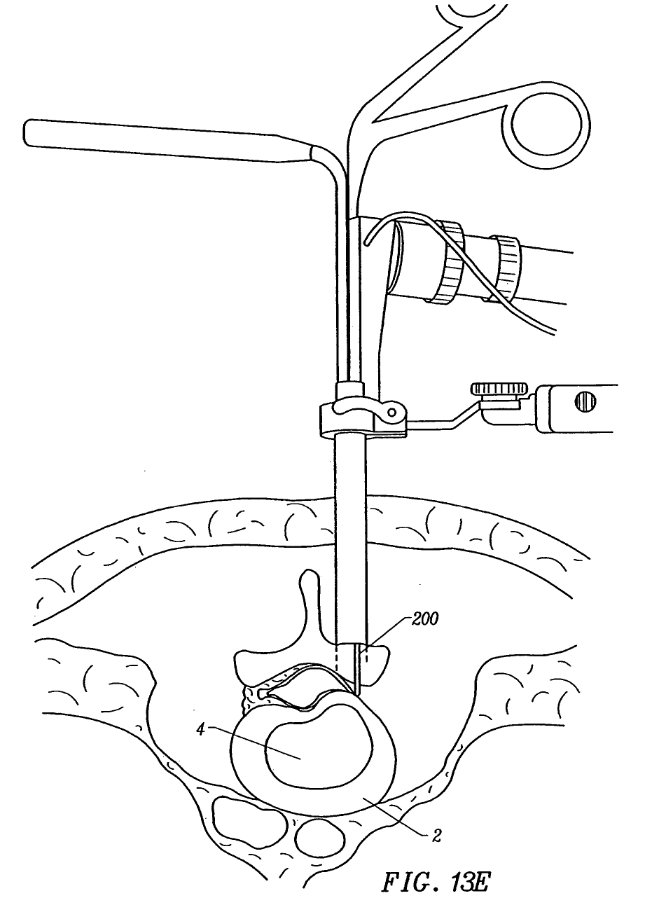

Prior to describing the inventive method and use of the inventive device, a basic discussion of the procedures used to access a herniated disc follows. FIG. 13A illustrates a herniated disc 2 having a nucleus pulpous 4. As illustrated, the herniated disc 2 impinges on a portion of the spinal cord or nerve root 6 which may cause pain as discussed above.

Although the present invention is particularly useful in micro or endoscopic discectomy procedures, e.g., for decompressing a nerve root with a lumbar discectomy, the invention may also be useful in open surgical or minimally invasive procedures. For instance, the probe 20 can be percutaneously introduced posteriorly through the patient's back directly into the spine.

As shown in FIG. 13B, a penetration 10 is made in the patient's back 12 so that the superior lamina 14 can be accessed. Typically, a small needle (not shown) is used initially to localize the disc space level, and a guidewire (not shown) is inserted and advanced under lateral fluoroscopy to the inferior edge of the lamina 10. Sequential cannulated dilators 16 are inserted over the guide wire and each other to provide a hole from the incision 10 to the lamina 14. As shown in FIG. 13C tubular retractor 18 is inserted to establish an operating corridor. Next, an endoscope 28 is then inserted into the tubular retractor 18 and the endoscope 28 is eventually secured (e.g., via a ring clamp 22.) Typically, soft tissue, muscle or other types of tissue are removed if they enter into the operative corridor as the dilators 16 and tubular retractor 18 advance to the lamina 14. Mechanical instruments, such as pituitary rongeurs, curettes, graspers, cutters, drills, microdebriders, and the like are used to remove the tissue. Unfortunately, these mechanical instruments greatly lengthen and increase the complexity of the procedure. In addition, these instruments sever blood vessels within this tissue, usually causing profuse bleeding that obstructs the surgeon's view of the target site. To alleviate these problems, the tissue may also be removed by electrosurgical probes such as those provided by Arthrocare Corp. of Sunnyvale, Calif.

Eventually, the probe 20 advances through the operative corridor. It should be noted that electrically conductive medium 113 may be provided through the access devices. Another advantage of the present invention is the ability to precisely ablate soft tissue without causing necrosis or thermal damage to the underlying and surrounding tissues, nerves or bone. In addition, the voltage can be controlled so that the energy directed to the target site is insufficient to ablate the lamina 14 so that the surgeon can literally clean the tissue off the lamina 14, without ablating or otherwise effecting significant damage to the lamina.

Referring now to FIGS. 13C and 13D, once the operating corridor is sufficiently cleared, a laminotomy and medial facetectomy is accomplished either with conventional techniques (e.g., Kerrison punch or a high speed drill) or with an electrosurgical probe 20 as discussed above. After the nerve root is identified, retraction can be achieved with a retractor 26. If necessary, epidural veins are cauterized either automatically or with the coagulation mode of the present invention. As a result, the annulus 30 of the disc 2 is exposed.

FIG. 13E illustrates a needle 200 being advanced to the annulus 30 of the disc 2. The invention is not limited to the needle shown, instead, the invention may be used in conjunction with many commercially available needles, including, but not limited to a Crawford type (e.g., a needle with bevel at the tip), a trocar type (e.g., a needle having a cutting tip and blunt cannula), and a taper type needle (e.g., a needle having a tip shaped like a pencil tip and blunt cannula.)

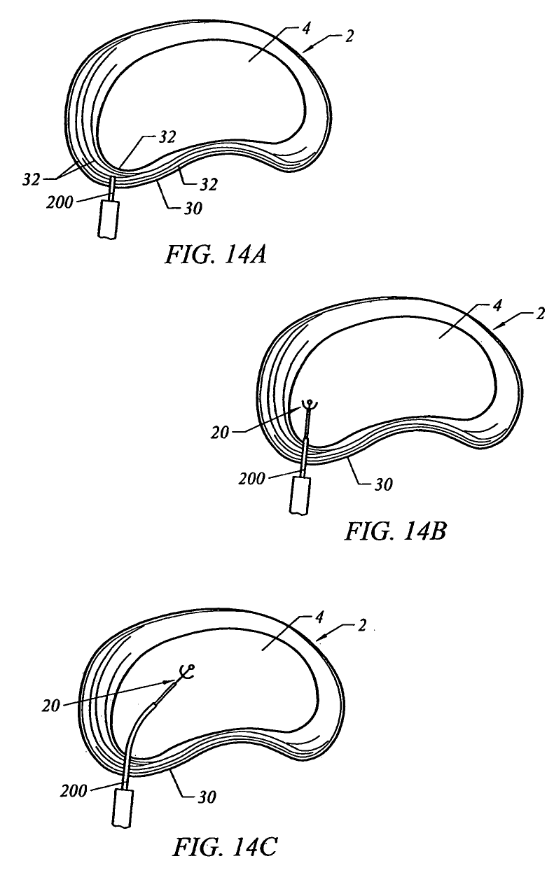

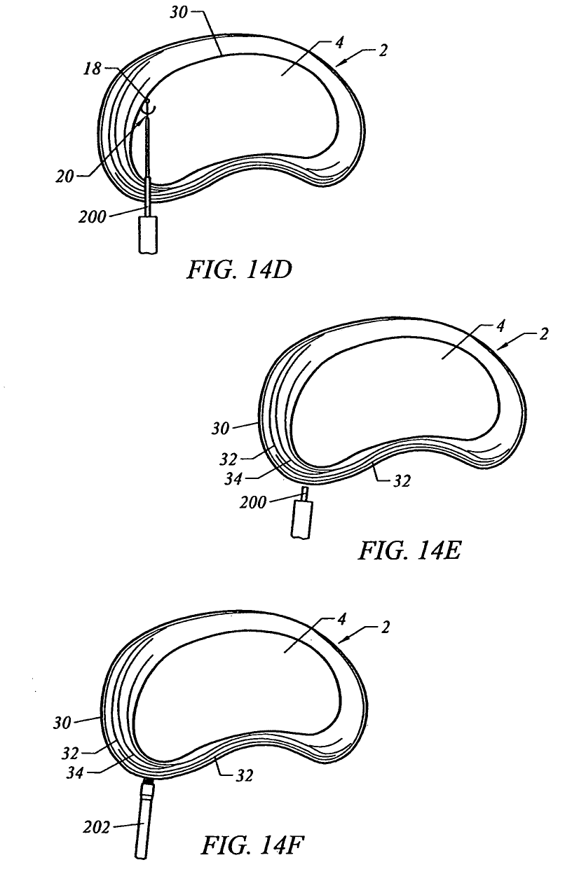

FIGS. 14A-14F illustrate the inventive method. As discussed above, the annulus fibrosus 30 forms the outer boundary of the disc and is composed of highly structured collagen fibers embedded in amorphous base substance also composed of water and proteoglycans. However, the amorphous base of the annulus 30 comprises collagen fibers that are arranged in concentric laminated bands 32. As the needle 200 advances into the annulus 30, it separates the laminated bands 32. It should be noted that the needle 200 may have a sharp tip with a taper that serves to separate the laminated bands 32, or the needle 200 may be blunt whereby the laminated bands are separated via blunt dissection.

In any event, the opening created by the needle 200 into the annulus 30 is minimal and does not rely on removing a significant amount of annulus 30 material. As a result, when the devices are subsequently removed from the annulus 30, the laminated bands 32 relax independently of one another and re-orient. This re-orientation of the laminated bands 32 closes the passageway created by the needle because the passageway formed by the separated bands 32 are no longer in alignment. Essentially, individual movement of the bands 32 dissipates the opening. As described hereafter, heat may be applied either prior to or subsequent to insertion of the needle 200 or even after removal of the needle 200. The heat is intended to cause shrinkage of the collagen fibers to increase movement of the fibers to aid in closure of the opening. Applicant believes that the inventive procedure creates an opening within a disc 2 that does not significantly compromise the integrity of the disc upon closure of the opening. A disc 2 having undergone the inventive procedure is believed to have an annulus 30 with a higher integrity than that of an annulus having undergone an annulotomy or a disc in which material was excised from the annulus.

Furthermore, as described above, conventional procedures require removal of excess nucleus material to relieve the pressure on a weakened annulus. However, because the inventive procedure leaves the annulus 30 of the disc with a higher integrity, it may not be necessary to remove as much nucleus material than if the annulus integrity was significantly compromised. Accordingly, the surgeon may be able to remove less nucleus material than would otherwise be desirable.

FIG. 14B illustrates advancement of a probe 20 into the nucleus pulposus 4 of the disc 2. While the inventive procedure is not limited to any particular bi-polar electrosurgical probe, a variation of the electrosurgical probe of the present invention provides additional benefits to allow minimizing the size of the entry into the annulus 30 of the disc 2. As illustrated in FIG. 14B, a probe having electrodes that are adapted to assume a reduced profile and an expanded profile provides the benefit of being able to enter the disc 2 through a minimum size opening, and when inserted into the nucleus pulposus 4, expand to ablate or coagulate a greater amount of tissue. One benefit of such a feature is that the speed of the procedure may be increased.

Once the probe 20 enters the disc 2 and/or nucleus 4, the probe 20 forms voids or channels within the disc 2 via ablation, and thermal energy may also be applied to the tissue surface immediately surrounding these voids or channels to cause thermal damage to the tissue surface, thereby stiffening and debulking the surrounding tissue structure of the disc. Applicant has discovered that such stiffening of the tissue structure in the disc helps to reduce the pressure applied against the spinal nerves by the disc, thereby relieving back and neck pain.

Electrically conductive medium is delivered to the target site, as described herein. Alternatively, the conductive medium is applied to the target site, or the distal end of probe 20 is dipped into conductive medium or gel prior to introducing the probe 2 into the patient. Moreover, the natural saline content of the disc may be sufficient such that an additional conductive medium or medium is not required. As noted above, electrically conductive medium may be introduced through a separate fluid delivery device. In such a case, the delivery device may also be introduced into the disc. The power supply is then activated and adjusted such that a high frequency voltage difference is applied to the electrode assembly as described above.

The invention may include pre-heating electrically conductive medium to a controlled temperature. An exemplary biocompatible fluid is isotonic saline. For procedures aimed at contraction of target tissue via shrinkage of collagen fibers within the tissue, the fluid is typically heated to a controlled temperature in the range of about 45° C. to 90° C., and more typically in the range of about 60° C. to 75° C. In one variation, the fluid may be heated to, and maintained at, the controlled temperature using a common fluid source unit. The fluid may be held at the controlled temperature in a fluid reservoir. The preheated fluid may be provided by the fluid source unit to a fluid delivery unit via one or more pumps and/or valves. The fluid delivery unit may be integral with the electrosurgical probe, or may be on a separate device.

Depending on the procedure, the surgeon may translate or otherwise move the electrodes relative to the target disc tissue to form one or more voids, holes, channels, stripes, divots, craters, or the like within the disc. In addition, the surgeon may purposely create some thermal damage within these holes, or channels to form scar tissue that will stiffen and debulk the disc. In one variation, the physician axially translates the electrode assembly into the disc tissue as the tissue is volumetrically removed to form one or more holes therein. The holes will typically have a diameter of less than 15 mm, preferably around 5 mm. Applicant has found that the present invention can quickly and cleanly create such holes, divots, or channels in tissue with the ablation technology described herein. A more complete description of methods for forming holes or channels in tissue can be found in U.S. Pat. No. 5,683,366, the complete disclosure of which is incorporated herein by reference for all purposes.

During the ablation process, the conductive medium between active and return electrodes generally minimizes current flow into the surrounding tissue, thereby minimizing thermal damage to the tissue. In some procedures, it may be desired to thermally damage the surface of the created opening to stiffen the tissue. For these reasons, it may be desired in some procedures to increase the thermal damage caused to the tissue surrounding the opening. Therefore, it may be necessary to either: (1) withdraw the probe slowly from the area being treated while coagulating the tissue. As discussed herein, coagulation is possible by passing electric current through the tissue surrounding the opening and creates thermal damage therein.

FIG. 14C illustrates advancing of the probe 20 along a curved path in the disc 2. The probe 20 has a distal portion that is bent to accomplish a curved trajectory. In other variations, the distal portion of shaft of the probe 20 comprises a flexible material which can be deflected relative to the longitudinal axis of the shaft. Such deflection may be selectively induced by mechanical tension of a pull wire, for example, or by a shape memory wire that expands or contracts by externally applied temperature changes. Moreover, a curved introducer needle 200 may be used to provide a curved trajectory. A more complete description of this embodiment can be found in U.S. Pat. No. 5,697,909, the complete disclosure of which has previously been incorporated herein by reference. Alternatively, needle or probe of the present invention may be bent by the physician to the appropriate angle using a conventional bending tool or the like.

FIG. 14D illustrates another advantage of the present invention. As described above, variations of the probe 20 comprise a return electrode tip portion 118. In probes of the present invention, the ratio of the return electrode surface area to the active electrode surface area will be sufficiently high to prevent the formation of a plasma layer immediately adjacent to the return electrode. Accordingly, ablation will not take place at the return electrode. Use of a probe 20 with a blunt return electrode tip portion 118 that extends beyond the active electrode permits advancement of the probe 20 to an inner wall of the annulus. Because ablation does not occur at the tip portion 118, the tip portion 118 may be used as a stop to prevent further advancement of the probe into the annulus 30, thereby preventing unintended damage to the annulus 30. Such a benefit allows removal of nucleus material without non-invasive imaging of the operative site.

Moreover, because of the above described configuration, the device may be used to approach the annulus wall without fear of penetrating the wall. As the device approaches the annulus, it may be used to provide heat to the annulus wall. Such a procedure may be used in an attempt to denervate nerve endings that are found within portions of the annulus. The attempt to denervate nerve endings in the annulus may be performed during and/or after the ablative process. It is readily apparent that an advantage of the present inventive device is that a single device may both ablate tissue and apply heat to denervate the annulus.

FIG. 14E illustrates the disc 2 after removal of the devices from the annulus 30. It is noted that the herniation of the disc 2 is reduced or eliminated. As illustrated, the laminated bands 32 within the annulus 30 relax and begin to close the opening 34. As described above, heat may be applied to the area of the opening 34 to aid in closure. FIG. 14F illustrates application of a device 202 to apply heat to the area of the opening 34 subsequent to removal of the device from the annulus 30. The heat may be applied via RF, resistive, laser, etc., applications. It will be noted that the heat may be applied to an area outside of the disc, or on the outside of the disc, and adjacent (or near) to the opening. Alternately, or in addition, heat may be applied to the interior surface of the opening.

Although the invention has been described primarily with respect to the treatment of intervertebral discs, it is to be understood that the methods and apparatus of the invention are also applicable to the treatment of other tissues, organs, and bodily structures by use of concepts, aspects, and embodiments that apply thereto. Thus, while the exemplary embodiments of the present invention have been described in detail, by way of example and for clarity of understanding, a variety of changes, adaptations, and modifications will be obvious to those of skill in the art. The scope of the present invention is limited solely by the appended claims.

The invention herein is described by examples and a desired way of practicing the invention is described. However, the invention as claimed herein is not limited to that specific description in any manner. Equivalence to the description as hereinafter claimed is considered to be within the scope of protection of this patent.

What is claimed is:

1. A method for treating an intervertebral disc comprising:
advancing an access device into the disc by separating layers of a fibrous outer portion of the disc to create a passageway into the disc with the access device;
advancing a treatment device into the disc using the access device; and
activating the treatment device to treat the disc;
wherein upon removal of the accessing device the separated layers of the fibrous outer portion substantially relax to remove the passageway.

2. The method of claim 1, wherein advancing an access device into the disc includes cutting a portion of the layer of the fibrous outer portion of the disc.

3. The method of claim 1, wherein advancing the access device comprises inserting a tapered introducer device into the fibrous outer portion of the disc.

4. The method of claim 1, wherein advancing the access device comprises inserting an introducer needle into the fibrous outer portion of the disc.

5. The method of claim 1, wherein the treatment device includes at least one active electrode and a return electrode, wherein activating the treatment device comprises applying a high frequency voltage between the active and return electrodes.

6. The method of claim 5, further comprising providing a conductive medium between the active and return electrodes to form a current path therebetween.

7. The method of claim 5, wherein advancing the treatment device comprises advancing the treatment device into a nucleus pulposus of the disc.

8. The method of claim 5, wherein activating the treatment device comprises ablating tissue within the disc.

9. The method of claim 5, wherein activating the treatment device comprises coagulating tissue within the disc.

10. The method of claim 1, further comprising expanding a portion of the treatment device prior to activating the treatment device.

11. The method of claim 1, further comprising advancing the treatment device until a portion of the treatment device contacts a fibrous inner portion of the disc.

12. The method of claim 11, wherein advancing the treatment device comprises using the fibrous inner portion of the disc to stop advancement of the treatment device.

13. The method of claim 11, further comprising applying heat to the fibrous inner portion of the disc to denervate a portion of the disc containing nerve endings.

14. The method of claim 1, further comprising inserting a scope adjacent to the disc prior to advancing the access device.

15. The method of claim 1, wherein advancing the treatment device comprises advancing the treatment device along a curved path into the disc.

16. The method of claim 15, wherein advancing the treatment device along a curved path comprises using a curved introducer to advance the treatment device.

17. The method of claim 1, further comprising performing non-invasive imaging prior to or during activating the treatment device.

18. The method of claim 17, wherein the non-invasive imaging comprises an imaging selected from the group consisting of fluoroscopy, x-ray, magnetic resonance imaging, and computed tomography.

19. The method of claim 1, further comprising applying energy to the fibrous outer portion of the disc.

20. The method of claim 19, where applying energy to the fibrous outer portion of the disc comprises applying energy adjacent to a site wherein advancing the access device into the disc occurs.

21. The method of claim 20, where applying energy comprises applying energy prior to removing the access device.

22. The method of claim 20, where applying energy comprises applying energy subsequent to removing the access device.

23. The method of claim 20, where applying energy comprises applying energy during removing the access device.

24. The method of claim 1, where the treatment device comprises a stop-portion adapted to prevent advancement of the treatment device into the disc.

25. The method of claim 24, wherein the stop-portion of the treatment device is located on a distal portion of the device.

26. The method of claim 24, wherein the stop-portion of the treatment device is adapted to prevent advancement of the treatment device into an inner wall of an annulus of the disc.

27. An electrosurgical device for use with a high-frequency power supply, the device comprising:
a shaft having a proximal portion and a distal portion;
a return electrode at the distal portion of the shaft and having a return electrode surface area, the return electrode distally terminating in a tip portion;
at least one active electrode at the distal portion of the shaft, and having an active electrode surface area, the active electrode further comprising an arm portion being radially spaced from the return electrode, wherein the tip portion of the return electrode is distally spaced from the arm portion of the active electrode; and
a connector located at the proximal portion of the shaft and adapted to couple the return electrode and each active electrode to respective poles of the high-frequency power supply.

28. The electrosurgical device of claim 27, wherein the return electrode surface area is greater than the active electrode surface area.

29. The electrosurgical device of claim 27, wherein at least part of the tip portion comprises a shape selected from a group consisting of a sphere, a semi-sphere, oblate sphere, and prolate sphere.

30. The electrosurgical device of claim 27, wherein a portion of the return electrode comprises at least one segment having a raised surface, whereby the raised surfaces increase the return electrode surface area.

31. The electrosurgical device of claim 30, wherein the at least one segment having the raised surface comprises a coil adjacent to the tip portion.

32. The electrosurgical device of claim 27, wherein the at least one active electrode comprises at least a first and a second active electrode.

33. The electrosurgical device of claim 32, wherein the first and second active electrodes are spaced 180 degrees on the device.

34. The electrosurgical device of claim 32, wherein a span between the arm portion of the first active electrode and the arm portion of the second active electrode is at least 3 mm.

35. The electrosurgical device of claim 27, wherein at least the arm portion of the active electrode is adapted to deform such that the device may assume a reduced profile.

36. The electrosurgical device of claim 35, wherein at least the arm portion of the active electrode is elastically deformable.

37. The electrosurgical device of claim 35, wherein at least a portion of the active electrode comprises a shape memory alloy such that the arm portion of the active electrode may return from the reduced profile upon application heat.

38. The electrosurgical device of claim 35, further comprising an electrode support physically connecting the return electrode to at least one of the active electrodes.

39. The electrosurgical device of claim 38, wherein the return electrode and the active electrode are moveable relative to each other.

40. The electrosurgical device of claim 27, further comprising an outer covering slidably moveable over the shaft and active electrode, and having an internal opening having a dimension smaller than a maximum radial distance from the active electrode arm portion to the return electrode.

41. The electrosurgical device of claim 27, wherein at least the active electrode arm portion comprises at least one section of reduced surface area adapted to produce a high current density.

42. The electrosurgical device of claim 27, wherein at least the active electrode arm portion comprises a cross-sectional shape selected from the group consisting of a d-shape, a square shape, a rectangular shape, a triangular shape, a circular shape, and an oval shape.

43. The electrosurgical device of claim 27, further comprising a hub at a proximal end of the shaft, wherein the connector comprises a cable and is integral to the hub.

44. The electrosurgical device of claim 27, wherein the hub comprises a handle.

45. An electrosurgical system for treating tissue with a high-frequency power supply, the system comprising:
a source of electrically conductive medium;
an electro surgical device for use with the high-frequency power supply, the device comprising,
a shaft having a proximal portion and a distal portion;
a return electrode at the distal portion of the shaft and having a return electrode surface area, the return electrode having a tip portion;
at least one active electrode at the distal portion of the shaft, and having an active electrode surface area, the active electrode further comprising an arm portion being radially spaced from the return electrode, wherein the tip portion of the return electrode is distally spaced from the arm portion of the active electrode; and
a connector located at the proximal portion of the shaft and adapted to couple the return electrode and each active electrode to respective poles of the high-frequency power supply; and
wherein the source of electrically conductive medium provides an electrically conductive medium which completes a circuit between the return electrode and the active electrode.

46. The method of claim 1, wherein the passageway is completely removed.
